(19) United States
(12) Patent Application Publication
(10) Pub. No.: US 2009/0163549 A1
(43) Pub. Date: Jun. 25, 2009

(54) Pharmaceutical Composition Comprising an Amide Derivative

(76) Inventor: Hiroyuki Kai, Osaka-fu (JP)

Correspondence Address: WENDEROTH, LIND & PONACK, L.L.P., 1030 15th Street, N.W.,, Suite 400 East, Washington, DC, 20005-1503, US

(21) Appl. No.: 12/085,941

(22) PCT Filed: Dec. 13, 2006
(86) PCT No.: PCT/JP2006/325313

§ 371 Date: Jun. 3, 2008

(30) Foreign Application Priority Data
Dec. 15, 2005 (JP) 2005-361643

(51) Int. Cl. A61K 31/167; A61K 31/4433; A61K 31/4439; A61K 31/4245; A61K 31/426; A61K 31/421; A61K 31/42; A61K 31/357; C07D 405/12; C07D 271/07; C07D 277/10; C07D 263/06; C07D 261/04; C07D 233/02; C07D 319/06; C07C 237/28; C07C 235/84

(52) U.S. Cl. 514/336; 514/342; 514/364; 514/365; 514/374; 514/380; 514/452; 514/467; 514/619; 514/621; 546/282.4; 548/132; 548/146; 548/215; 548/243; 548/300.1; 549/373; 564/164; 564/169

(57) ABSTRACT

The present invention provides a novel modulator of the TRPV1-receptor function, comprising a compound of the formula; wherein ring A is an optionally substituted carbocycle or heterocycle, ring B is an optionally substituted benzene ring or an optionally substituted 6-membered heteroaromatic ring containing N atom, a dashed line means existence or absence of a bond, n is 1 or 2, R¹ and R² are hydrogen etc., R³ is lower alkyl, R⁴ is lower alkyl or aryloxy, or R³ and R⁴ taken together may form optionally substituted 5- or 6-membered non-aromatic heterocycle.

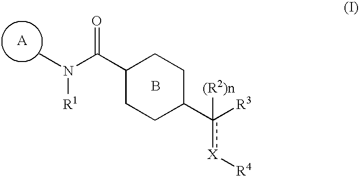

### TECHNICAL FIELD

The present invention relates to an amide derivative and a pharmaceutical composition comprising the said derivative as an active ingredient. In details, it relates to an aromatic carboxylic acid amide derivative and a modulator of the TRPV1-receptor function (e.g., an antagonist of the TRPV1 receptor).

### BACKGROUND ART

It has long been known that capsaicin of a pungent substance is a kind of pain transmitters. However, a receptor gene of capsaicin was cloned recently and demonstrated that it is an ionotropic type of receptor which responds three different noxious stimuli of capsaicin, an acid and heat over 43° C. The receptor was named as Vanilloid Receptor 1 (VR1) since capsaicin and resiniferatoxin, a powerful analog of capsaicin, shared a common vanillyl moiety.

Later, it was found that VR1 belonged to a larger TRP ion channel super family which includes TRPM, TRPA and the like, and VR1 was renamed as TRPV1. At present, TRPV1 forms a TRPV subfamily together with other five subtypes of TRPV2-TRPV6.

With respect to an endogenous ligand for TRPV1, anandamide is known as one of the candidates. It is also known that TRPV1 is activated (a threshold value of temperature is lowered) at near the body temperature under a weakly acidic condition or in the presence of mediators related to inflammation (e.g., ATP or bradykinin), while pH value is lowered and such mediators are produced around the inflammatory site.

Some antagonists for TRPV1 have been already reported and it is considered that their analgesic activity is brought by antagonizing TRPV1-activation resulted from an endogenous TRPV1 ligand, acidification of the inflammatory site and/or a lowered threshold value of temperature.

Besides, capsaicin is known to have an analgesic activity as well as an algesic activity. The analgesic activity results from reduced responsibility of the terminal of sensory neuron to noxious stimuli after exposure to a TRPV1 agonist such as capsaicin and the like, and TRPV1 agonists as well as its antagonists may be used as an analgesic agent.

For example, PCT International Gazette (WO2004/110986) discloses benzamide derivatives useful as an inhibitor of TRPV1-activation, but derivatives substituted with a non-aromatic heterocyclyl or oxime group at the para-position of the said benzoic acid are not disclosed.

Besides, other PCT Gazette (WO2004/056774) discloses biphenyl carboxylic acid derivatives and its analogues, and their activities as a modulating factor of capsaicin receptors. Moreover, other PCT Gazette (WO2004/069792) describes that some quinolinamide derivatives may act as a modulating factor of capsaicin receptors.

However, none of these TRPV1 antagonists is sufficient for an analgesic agent and a new TRPV1 antagonist and/or modulator of TRPV1-receptor function is still necessary.

### DISCLOSURE OF INVENTION

The objective of the present invention is to provide a modulator of TRPV1-receptor function, especially TRPV1 antagonist, having a novel chemical structure.

The inventor has found that

(1) para-substituted benzamide derivatives of the formula (I);

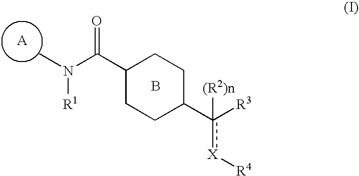

wherein ring A is an optionally substituted monocyclic or bicyclic carbocycle, or an optionally substituted monocyclic or bicyclic heterocycle,
ring B is an optionally substituted benzene ring or an optionally substituted 6-membered heteroaromatic ring containing N atom,
R¹ is hydrogen, optionally substituted lower alkyl or optionally substituted acyl,
a dashed line means existence or absence of a bond,
1) when the dashed line means existence, then n is 0, and
1-1) X is ═CR^{x}— or ═N—, and R³ and R⁴ are taken together, with the atoms to which they are bonded, to form an optionally substituted 5-membered non-aromatic heterocycle or optionally substituted 6-membered non-aromatic heterocycle containing at least one heteroatom selected from O and S, and Rx is hydrogen, halogen, lower alkyl, halogeno(lower)alkyl, lower alkoxy, halogeno(lower)alkoxy or acyl, or
1-2) X is ═N—, R³ is lower alkyl, R⁴ is lower alkoxy or aryloxy,
2) when the dashed line means absence of a bond, then n is 1, R² is hydrogen or optionally substituted lower alkyl, X is —O—, —S— or —NR⁵—, and R³ and R⁴ taken together, with the atoms to which they are bound, to form optionally substituted 5-membered non-aromatic heterocycle or optionally substituted 6-membered non-aromatic heterocycle containing at least one heteroatom selected from O and S, R⁵ is hydrogen, lower alkyl, halogeno(lower)alkyl, alkoxy, halogeno(lower)alkoxy or acyl,
a pharmaceutically acceptable salt or solvate thereof may act as a modulator of TRPV1-receptor function, especially a TRPV1-antagonist and completed the present invention.

In addition, the present invention provides

(2) the pharmaceutical composition for modulating TRPV1-receptor function according to (1) above, wherein

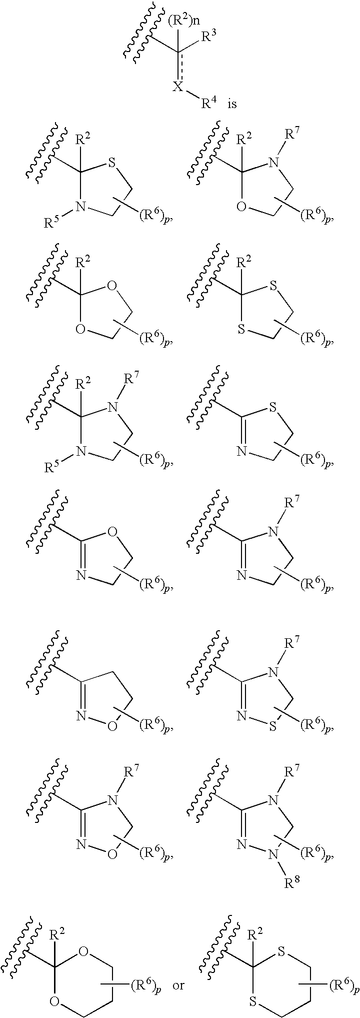

wherein R² is hydrogen or lower alkyl, p is an integer of 0 to 4, R⁶ is hydrogen, halogen, lower alkyl, lower alkoxy, halogeno(lower)alkyl, halogeno(lower)alkoxy or acyl when R⁶ bounds to a constituent carbon atom of the ring, when p is 2 or more, two of R⁶ taken together may form oxo to attach to a constituent carbon atom or a constituent sulfur atom,
two of R⁶ taken together may form thioxo to attach to a constituent carbon atom,
each of R⁶ may be independently different, and
R⁷ and R⁸ are each independently hydrogen, lower alkyl, lower alkoxy, halogeno(lower)alkyl, halogeno(lower)alkoxy or acyl;
(3) the pharmaceutical composition for modulating TRPV1-receptor function according to (1) or (2) above, wherein ring A is optionally substituted benzene, optionally substituted naphthalene, optionally substituted tetrahydronaphthalene, optionally substituted pyridine, optionally substituted pyrimidine, optionally substituted pyridazine, optionally substituted pyrazine, optionally substituted pyrrole, optionally substituted pyrazole, optionally substituted imidazole, optionally substituted triazole, optionally substituted oxazole, optionally substituted isoxazole, optionally substituted thiazole, optionally substituted isothiazole, optionally substituted indole, optionally substituted isoindole, optionally substituted indoline, optionally substituted isoindoline, optionally substituted benzimidazole, optionally substituted benzimidazoline, optionally substituted quinoline, optionally substituted isoquinoline or optionally substituted tetrahydroquinoline;
(4) the pharmaceutical composition for modulating TRPV1-receptor function according to (1) or (2) above, wherein ring A is para-substituted benzene;
(5) the pharmaceutical composition for modulating TRPV1-receptor function according to any one of (1) to (4), wherein R¹ is hydrogen;
(6) the pharmaceutical composition for modulating TRPV1-receptor function according to any one of (1) to (5), wherein ring B is optionally substituted benzene or optionally substituted pyridine;
(7) a compound of the formula (I′);

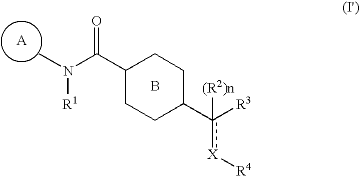

wherein each of symbols is the same as defined in (1), with the proviso that when ring A has substituent(s), then the substituent(s) is(are) at least one group selected from the group of halogen, hydroxy, lower alkyl, halogeno(lower)alkyl, hydroxy lower alkyl, lower alkoxy, halogeno(lower)alkoxy, hydroxy(lower)alkoxy, amino, lower alkylamino, acylamino, cycloalkyl, aryl, aryloxy and heterocyclyl,
when the optionally substituted 5-membered non-aromatic heterocycle or optionally substituted 6-membered non-aromatic heterocycle containing at least one heteroatom selected from O and S which are formed by R³ and R⁴ taken together, with the atoms to which they are bound, has substituent(s), then the substituent(s) is(are) at least one of group selected from the group of halogen, lower alkyl, halogeno(lower)alkyl, lower alkoxy, halogeno(lower)alkoxy, acyl, oxo and thioxo, and then the non-aromatic heterocycle is not dithiolane, a pharmaceutically acceptable salt or a solvate thereof;
(8) the compound, the pharmaceutically acceptable salt or the solvate thereof according to (7) above, wherein R³ and R⁴ taken together, with the atoms to which they are bound, to form optionally substituted 5-membered non-aromatic heterocycle;
(9) a compound of the formula (I′″):

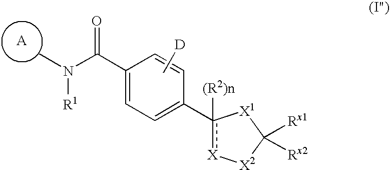

wherein R¹ is hydrogen, optionally substituted lower alkyl or optionally substituted acyl,

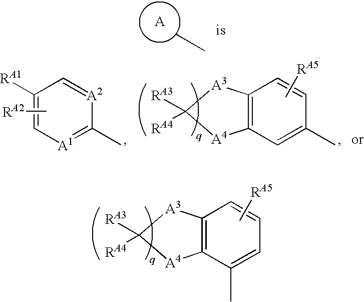

### A¹ is N or CR^{A6}, A² is N or CR^{A7}

### A³ is NR^{A8} or CR^{A9}R^{A10}, A⁴ is NR^{A11} or CR^{A12}R^{A13},

R^{A1} is lower alkyl, halogeno(lower)alkyl, halogeno(lower)alkoxy or halogen,
R^{A2}, R^{A3}, R^{A4}, R^{A5}, R^{A6}R^{A7}, R^{A8}, R^{A9}, R^{A10} CR^{A11}, R^{A12} A^{A13} are each independently hydrogen, halogen, hydroxy, lower alkyl, halogeno(lower)alkyl, hydroxy(lower)alkyl, lower alkoxy, halogeno(lower)alkoxy, hydroxy(lower)alkoxy, amino, lower alkylamino, acylamino, cycloalkyl, aryl, aryloxy or heterocyclyl,
each of R^{A3} or each of R^{A4} may be independently different,
q is an integer of 1 to 3,
D is hydrogen, halogen, lower alkyl, lower alkoxy or acyl, dashed line means existence or absence of a bond,
1) when the dashed line means existence of a bond, then n is 0, and
X is ═CR^{x}— or ═N—, Rx is hydrogen, halogen, lower alkyl, halogeno(lower)alkyl, lower alkoxy, halogeno(lower)alkyl or acyl;
2) when the dashed line means absence of a bond, then n is 1, R² is hydrogen or optionally substituted lower alkyl, X is —O—, —S— or —NR⁵—, R⁵ is hydrogen, lower alkyl, halogeno(lower)alkyl, lower alkoxy, halogeno(lower)alkoxy or acyl,
X¹ and X² are each independently —O—, —S—, —NR⁵— or —CR^{x3}R^{x4}—, R^{x1}, R^{x2}, R^{x3} and R^{x4} are each independently hydrogen, halogen, lower alkyl, halogeno(lower)alkyl, lower alkoxy, halogeno(lower)alkoxy or acyl, R^{x1} and R^{x2}, or R^{x3} and R^{x4} taken together may form oxo or thioxo,
each of R⁵, each of R^{x3}, or each of R^{x4} may be independently different,
a pharmaceutically acceptable salt or a solvate thereof;
(10) a pharmaceutical composition comprising the compound, the pharmaceutically acceptable salt or the solvate thereof according to any one of (7) to (9) as an active ingredient;
(11) a pharmaceutical composition for modulating TRPV1-receptor function comprising the compound, the pharmaceutically acceptable salt or the solvate thereof according to any one of (7) to (9) as an active ingredient;
(12) the pharmaceutical composition for modulating TRPV1-receptor function according to any one of (1) to (6) and (11), which is used as a TRPV1-receptor antagonist;
(13) the pharmaceutical composition for modulating TRPV1-receptor function according to any one of (1) to (6), (11) and (12), which is used as an analgesic agent;
(14) a method of modulating TRPV1-receptor function characterized by administrating the modulator of TRPV1-receptor function according to any one of (1) to (6), (11) and (12);
(15) Use of the compound, the pharmaceutically acceptable salt or the solvate thereof according to any one of (1) to (6), (11) and (12), for manufacturing of a medicament to modulate TRPV1-receptor function;
(16) a method to treat or relieve a pain characterized by administrating the modulator of TRPV1-receptor function according to any one of (1) to (6), (11) and (12); and
(17) use of the compound, the pharmaceutically acceptable salt or the solvate thereof according to any one of (1) to (6), (11) and (12), for manufacturing of an analgesic agent.

The term, “monocyclic or bicyclic carbocycle”, as used herein, encompasses “monocyclic or bicyclic aromatic carbocycle”, “C3-C8 monocyclic non-aromatic carbocycle”, benzene fused with “C3-C8 monocyclic non-aromatic carbocycle”, and carbocycle formed by fusing two “C3-C8 monocyclic non-aromatic carbocycle”s. The ring A—N bond may attach at any ring when the fused ring is bicyclic.

Concrete examples of “monocyclic or bicyclic carbocycle” include benzene and naphthalene as “monocyclic or bicyclic aromatic carbocycle”, cyclopropane, cyclobutane, cyclopentane, cyclohexane, cycloheptane, cyclooctane, cyclopentene, cyclohexene and cycloheptene as “C3-C8 monocyclic non-aromatic carbocycle”, and indane, indene, pentalene, tetrahydronaphthalene and the like.

The term, ““monocyclic or bicyclic heterocycle” encompasses “5- or 6-membered monocyclic aromatic heterocycle”, “3- to 6-membered non-aromatic heterocycle”, “5- or 6-membered aromatic heterocycle fused with other ring” and “3- to 6-membered non-aromatic heterocycle fused with other ring”.

The term, “other ring”, as used herein, refers to a benzene ring, “C3-C8 monocyclic non-aromatic carbocycle”, “5- or 6-membered monocyclic aromatic heterocycle” or “3- to 6-membered non-aromatic heterocycle”. The ring A—N bond may attach at any ring when the fused ring is bicyclic.

Examples of “5- or 6-membered monocyclic aromatic heterocycle” include pyrrole, imidazole, pyrazole, pyridine, pyridazine, pyrimidine, pyrazine, triazole, triazine, tetrazole, isoxazole, oxazole, oxadiazole, isothiazole, thiazole, thiadiazole, furan, thiophene and the like.

Examples of “3- to 6-membered non-aromatic heterocycle” include dioxane, thiirane, oxirane, oxathiorane, azetidine, thiane, pyrrolidine, pyrroline, imidazolidine, imidazoline, pyrazolidine, pyrazoline, piperidine, piperazine, morpholine, thiomorpholine, dihydropyridine, tetrahydrofuran, tetrahydropyran, tetrahydrothiazoline, tetrahydroisothiazoline and the like.

Examples of “5- or 6-membered aromatic heterocycle fused with other ring” and “3- to 6-membered non-aromatic heterocycle fused with other ring” include indole, isoindole, indazole, indolizine, indoline, isoindoline, quinoline, isoquinoline, cinnoline, phthalazine, quinazoline, naphthyridine, quinoxaline, purin, pteridine, benzopyran, benzimidazole, benzisoxazole, benzoxazole, benzoxadiazole, benzisothiazole, benzothiazole, benzothiadiazole, benzofuran, isobenzofuran, benzothiophene, benzotriazole, imidazopyridine, triazolopyridine, imidazothiazole, pyrazinopyridazine, benzimidazoline, benzodioxane, quinazoline, quinoline, isoquinoline, naphthylidine, dihydropyridine, tetrahydroquinoline, tetrahydrobenzothiophen and the like.

Examples of “C3-C8 monocyclic non-aromatic carbocycle” include cyclopropane, cyclobutane, cyclopentane, cyclohexane, cycloheptane, cyclooctane, cyclopentene, cyclohexene, cycloheptene and the like.

Examples of a substituent in “optionally substituted monocyclic or bicyclic carbocycle”, “optionally substituted monocyclic or bicyclic heterocycle”, “an optionally substituted benzene ring”, “optionally substituted naphthalene”, “optionally substituted tetrahydronaphthalene”, “optionally substituted pyridine”, “optionally substituted pyrimidine”, “optionally substituted pyridazine”, “optionally substituted pyrazine”, “optionally substituted pyrrole”, “optionally substituted pyrazole”, “optionally substituted imidazole”, “optionally substituted triazole”, “optionally substituted oxazole”, “optionally substituted isoxazole”, “optionally substituted thiazole”, “optionally substituted isothiazole”, “optionally substituted indole”, “optionally substituted isoindole”, “optionally substituted indoline”, “optionally substituted isoindoline”, “optionally substituted benzimidazole”, “optionally substituted benzimidazoline”, “optionally substituted quinoline”, “optionally substituted isoquinoline” and “optionally substituted tetrahydroquinoline” include halogen, hydroxy, lower alkyl optionally substituted with a group α of substituents, lower alkenyl optionally substituted with a group α of substituents, lower alkoxy optionally substituted with a group α of substituents, acyl optionally substituted with a group α of substituents, acyloxy optionally substituted with a group α of substituents, carboxy, lower alkoxycarbonyl optionally substituted with a group α of substituents, amino optionally substituted with a group a of substituents, lower alkylamino optionally substituted with a group α of substituents, acylamino optionally substituted with a group α of substituents, carbamoyl, lower alkylcarbamoyl optionally substituted with a group a of substituents, arylcarbamoyl optionally substituted with a group β of substituents, cyano, nitro, cycloalkyl optionally substituted with a group β of substituents, cycloalkenyl optionally substituted with a group β of substituents, aryl optionally substituted with a group β of substituents, aryloxy optionally substituted with a group β of substituents, arylthio optionally substituted with a group β of substituents, heterocyclyl optionally substituted with a group β of substituents, heterocyclyloxy optionally substituted with a group β of substituents, and heterocyclylthio optionally substituted with a group β of substituents, and they can be substituted with one or more of substituents selected from these substituents.

The term, “a group α of substituents”, as used herein, refers to halogen, hydroxy, acyl, acyloxy, amino, lower alkylamino, acylamino, carboxy, a lower alkoxycarbonyl, cyano, nitro and aryl, and the term, “a group β of substituents” refers to halogen, hydroxy, lower alkyl, halogeno(lower)alkyl, lower alkoxy, halogeno(lower)alkoxy, acyl, acyloxy, amino, lower alkylamino, acylamino, carboxy, lower alkoxycarbonyl, cyano and nitro.

The expression, “optionally substituted with a group of substituents” means that it may be substituted with one or more of the substituent(s) selected from the group.

Examples of “6-membered heteroaromatic ring containing N atom” include a 6-membered aromatic ring comprising at least one nitrogen atom as a ring member such as pyridine, pyridazine, pyrimidine, pyrazine, 1,3,4-triazine and 1,3,5-triazine.

Substituents of “optionally substituted 6-membered heteroaromatic ring containing N atom” are the same as those of “optionally substituted monocyclic or bicyclic carbocycle” and may be substituted with one or more of the substituent(s) selected from the group described above.

Examples of “R³ and R⁴ taken together, with the atoms to which they are bound, to form optionally substituted 5-membered non-aromatic heterocycle or optionally substituted 6-membered non-aromatic heterocycle containing at least one heteroatom selected from O and S” include groups of the formula;

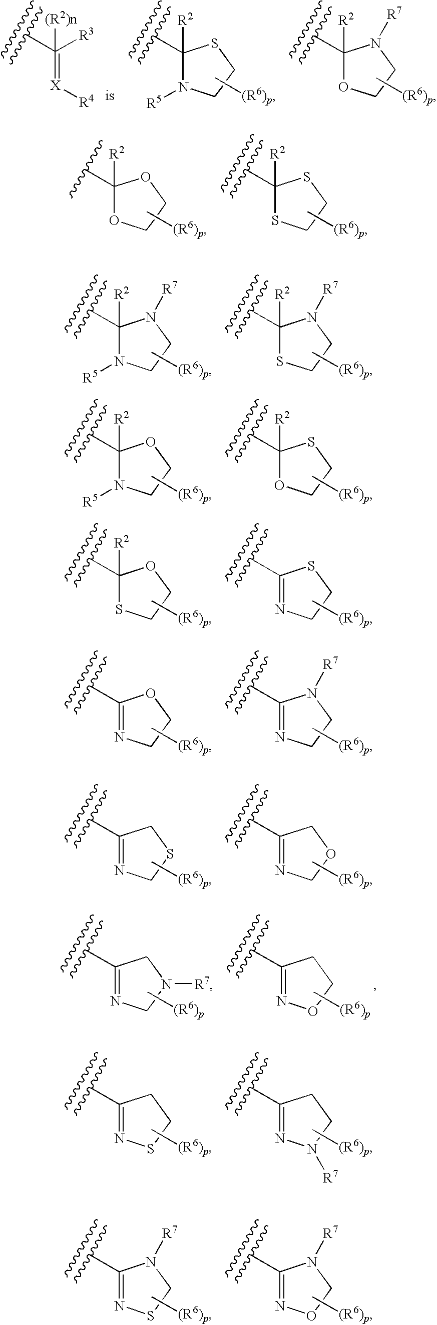

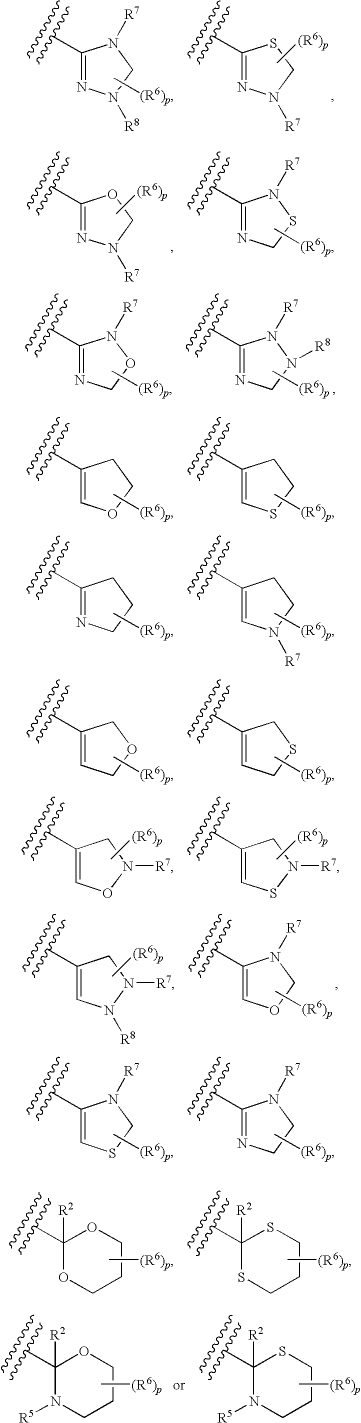

wherein the symbols are the same as defined above.

When p is 2 or more, two of R⁶ taken together may form oxo to attach to a constituent carbon atom or a constituent sulfur atom, or two of R⁶ taken together may form thioxo to attach to a constituent carbon atom. Examples of such cases include groups of the followings;

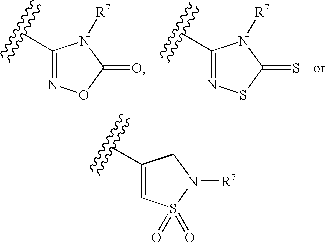

Examples of halogen include F, Cl and Br.

A moiety of halogen in halogeno(lower)alkyl and halogeno(lower)alkoxy is the same as the halogen above.

Examples of “lower alkyl” include straight or branched alkyl having 1 to 10, preferably 1 to 6, more preferably 1 to 3 carbon(s) such as methyl, ethyl, n-propyl, isopropyl, n-butyl, isobutyl, sec-butyl, tert-butyl, n-pentyl, isopentyl, neopentyl, hexyl, isohexyl, n-heptyl, isoheptyl, n-octyl, isooctyl, n-nonyl, n-decyl and the like. Methyl or ethyl is especially preferable.

Examples of substituents in “optionally substituted lower alkyl” include one or more of substituent(s) selected from the substituent group a described above.

An alkyl moiety of “halogeno(lower)alkyl”, “hydroxy(lower)alkyl”, “lower alkoxy”, “halogeno(lower)alkoxy”, “hydroxy(lower)alkoxy”, “lower alkylamino”, “lower alkylcarbamoyl” and “lower alkoxycarbonyl” is the same as that of “lower alkyl”.

Examples of “lower alkenyl” include straight or branched alkenyl containing one or more double bond at any position having 2 to 10, preferably 2 to 8, more preferably 3 to 6 carbon atoms, such as vinyl, allyl, propenyl, isopropenyl, butenyl, isobutenyl, prenyl, butadienyl, pentenyl, isopentenyl, pentadienyl, hexenyl, isohexenyl, hexadienyl, heptenyl, octenyl, nonenyl, decenyl and the like.

Examples of “acyl” include aliphatic acyl with 1 to 7 carbon atom(s) and aroyl, such as formyl, acetyl, propionyl, butyryl, isobutyryl, valeryl, pivaloyl, hexanoyl, acryloyl, propioloyl, metacryloyl, crotonoyl and benzoyl.

A moiety of acyl in “acyloxy” and “acylamino” is the same as that of acyl.

Examples of “optionally substituted acyl” include one or more of the substituent(s) selected from the substituent group α above when the acyl group is an aliphatic acyl, and more of the substituent(s) selected from the substituent group β above when the acyl group is aroyl.

Examples of “cycloalkyl” include carbocycle groups having 3 to 10, preferably 3 to 8, more preferably 4 to 8 carbon atoms, such as cyclopropyl, cyclobutyl, cyclopentyl, cyclohexyl, cycloheptyl, cyclooctyl, cyclononyl and cyclodecyl.

Examples of “cycloalkenyl” include non-aromatic carbocycles of the aforementioned “cycloalkyl” having one or more of double bond at any position on the ring.

Examples of “aryl” include phenyl, naphthyl, anthryl and phenanthryl, and phenyl is especially preferable.

A moiety of aryl in “aryloxy”, “arylcarbamoyl” and “arylthio” is the same as that of “aryl” above.

Examples of “heterocycle” includes heterocycle groups containing any one or more of heteroatom selected from O, N and S as a member of the ring, and specifically, a 5 or 6-membered heteroaryl such as pyrrolyl, imidazolyl, pyrazolyl, pyridyl, pyridazinyl, pyrimidinyl, pyrazinyl, triazolyl, triazinyl, tetrazolyl, isoxazolyl, oxazolyl, oxadiazolyl, isothiazolyl, thiazolyl, thiadiazolyl, furyl and thienyl; a fused bicyclic heterocycle group such as indolyl, isoindolyl, indazolyl, indolizinyl, indolinyl, isoindolinyl, quinolyl, isoquinolyl, cinnolinyl, phthalazinyl, quinazolinyl, naphthylidinyl, quinoxalinyl, purinyl, pteridinyl, benzopyranyl, benzimidazolyl, benzisoxazolyl, benzoxazolyl, banzoxadiazolyl, benzisothiazolyl, benzothiazolyl, benzothiadiazolyl, benzofuryl, isobenzofuryl, benzothienyl, benzotriazolyl, imidazopyridyl, triazolopyridyl, imidazothiazolyl, pyrazinopyridazinyl, quinazolinyl, quinolyl, isoquinolyl, naphthylidinyl, dihydropyridyl, tetrahydroquinolyl, and tetrahydrobenzothienyl; a fused tricyclic heterocycle group such as carbazolyl, acridinyl, xanthenyl, phenothiazinyl, phenoxathiinyl, phenoxazinyl and dibenzofuryl; and a non-aromatic heterocycle group such as dioxanyl, thiiranyl, oxiranyl, oxathioranyl; azetidinyl, thianyl, pyrrolidinyl, pyrrolinyl, imidazolidinyl, imidazolinyl, pyrazolidinyl, pyrazolinyl, piperidyl, piperazinyl, morpholinyl, morpholino, thiomorpholinyl, thiomorpholino, dihydropyridyl, tetrahydrofuryl, tetrahydropyranyl, tetrahydrothiazolyl and tetrahydroisothiazolyl. Preferred example is the 5 to 6-membered heteroaryl or the non-aromatic heterocycle group. The heterocycle moiety of “heterocyclyloxy” and “heterocyclylthio” are the same as that of “heterocycle” above.

As a salt of the present invention a pharmaceutically acceptable salt can be used, and examples of a base addition salt include an alkali metal salt such as a sodium salt and a potassium salt; an alkali earth metal salt such as calcium salt and a magnesium salt; an ammonium salt; an aliphatic amine salt such as a trimethylamine salt, triethylamine salt, a dicyclohexylamine salt, an ethanolamine salt, a diethanolamine salt, a trietanolamine salt and a procaine salt; an aralkylamine salt such as N,N-dibenzylethylenediamine salt; a heteroarylamine salt such as a pyridine salt, a picoline salt, a quinoline salt and a isoquinoline salt; a quaternary ammonium salt such as a tetramethylammonium salt, a tetraethylammonium salt, benzyltrimethylammonium salt, a benzyltriethylammonium salt, a benzyltributhylammonium salt, a methyltrioctylammonium salt and a tetrabutylammonium salt; and a basic amino acid salt such as an arginine salt and a lysine salt.

Examples of an acid addition salt include an inorganic acid salt such as hydrochloride, sulfate, nitrate, phosphate, carbonate, bicarbonate and perchlorate; an organic acid salt such as oxalate, acetate, propionate, lactate, maleate, fumarate, tartarate, malate, citrate and ascorbate; sulfonate such as methanesulfonate, isethionate, benzenesulfonate and p-toluenesulfonate; and an acidic amino acid salt such as aspartate and glutamate.

A compound (I) may be a solvate of water, acetonitrile, ethyl acetate, methanol, ethanol and the like. A solvation number of the compound of the present invention may vary depending upon the preparation process, purification method and/or condition of the crystallization etc., but usually one to five mole(s) of the solvent are included per one mole of the compound (I).

The compound of the present invention can be prepared, for example, according to the reaction scheme;

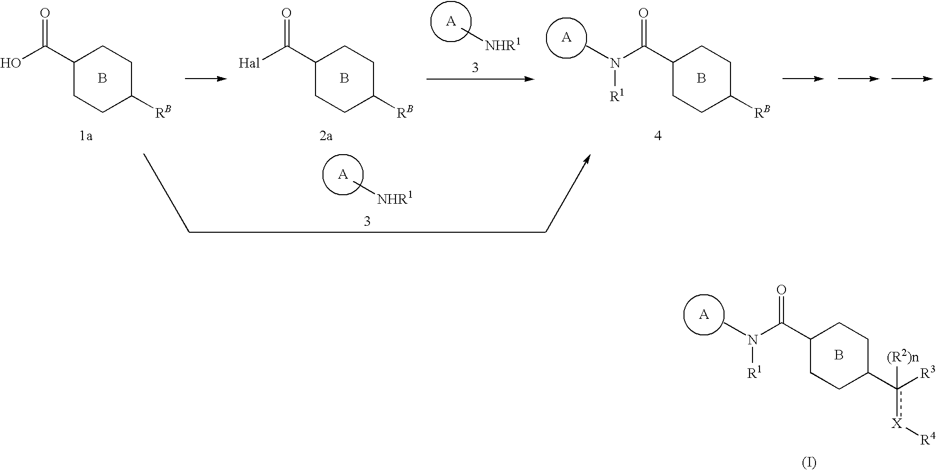

wherein R^{B} is formyl, cyano or acyl, Hal is halogen and other symbols are the same as described before.

The benzoic acid derivative 1a is reacted with a halogenating agent at about −20 to 100° C. to afford the acid halide 2a. The reaction is carried in a solvent such as dichloromethane, dichloroethane, chloroform, acetonitrile, tetrahydrofuran, dioxane, dimethoxyethane, benzene, toluene and the like. Examples of the halogenating agent include oxalyl chloride, thionyl chloride and the like, and dimethylformamide or the like may be added as a catalyst, if necessary.

The resulting acid halide 2a is reacted with the aniline derivative 3 in a suitable solvent under a base at about 0 to 50° C., preferably at about 15 to 30° C. for about 5 minutes to 10 hours, preferably for 30 minutes to 5 hours to afford the synthetic intermediate 4. The examples of the solvent include tetrahydrofuran, dimethylformamide, diethyl ether, dichloromethane, toluene, benzene, xylene, cyclohexane, hexane, chloroform, ethyl acetate, butyl acetate, pentane, heptane, dioxane, acetone, acetonitrile, water and the mixture thereof. The examples of the base include triethylamine, N-methylmorpholine, 4-dimethylaminopyridine and the like, and their mixture can be used as well as the single solvent. Thionyl chloride, acid halide, acid anhydride, activated ester and the like may be added as an activating agent, if necessary.

The synthetic intermediate 4 may also be prepared by reacting the benzoic acid derivative 1 with the aniline derivative 3 in a suitable solvent at about 0 to 50° C., preferably at about 15 to 30° C. for about 5 minutes to 10 hours, preferably for about 30 minutes to 5 hours using a condensing reagent. Examples of the solvent include dichloromethane, tetrahydrofuran, N,N-dimethylformamide and the like. Examples of the condensing reagent include water-soluble carbodiimide, dicyclohexylcarbodiimide, 1-(3-dimethylaminopropyl)-3-ethylcarbodiimide hydrochloride, N,N′-carbonyldiimidazole, ethyl chlorocarbonate, isobutyl chlorocarbonate, thionyl chloride, and oxalyl chloride.

Beside, an additive of condensation reaction such as 1-hydroxybenzotriazole, 3,4-dihydro-3-hydroxy-4-oxo-1,2,3-benzotriazine and the like may be added.

Next, the compound (I) can be prepared by cyclization of the R^{B} moiety or through transformation of the moiety into the oxime derivative using a conventional method. (See Examples Described Below.)

In addition, cyclization to the heterocycles may be achieved in reference to the following literatures.
R. E. Bolton et al., Tetrahedron Lett. 1995, 36, 4471
P. C. Shrum et al., J. Med. Chem. 1992, 35, 3691
H. Agirbas et al., Phosphorus, Sulfur Silicon Relat. Elem. 1992, 66, 321
J. Lessel et al., Pharmazie 2000, 55, 22
A. Mayence et al., Eur. J. Med. Chem. 2004, 39, 547
U. Azzena et al., Tetrahedron 2005, 61, 3177
WO 95/26956
C. D. Bedford et al., J. Med. Chem. 1986, 29, 2174
H. Kai et al., Heterocycles 2002, 57, 2299
J. G. Badiang et al., J. Org. Chem. 1996, 61, 2484
A. Goosen et al., J. Chem. Res., Synop. 1995, 394
A. J. Liepa et al., Aust. J. Chem. 1997, 50, 755
A. J. Showler et al., Chem. Rev. 1967, 67, 427
R. P. Hatch et al., J. Org. Chem. 1978, 43, 4172
V. K. Yadav et al., Tetrahedron Lett. 1988, 29, 897
E. P. Goldberg et al., J. Am. Chem. Soc. 1955, 77, 359
X. Fernandez, et al., Tetrahedron Lett. 2000, 41, 3381
M. DZURILLA, et al., Collect Czech Chem Commun 1999, 64, 1448.

Alternatively, the compound (I) may be prepared by using the compound 1b as shown in the next scheme;

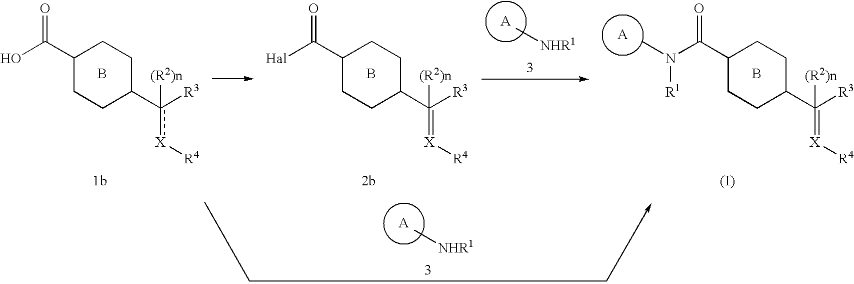

wherein each process may be carried out in the same manner described above.

The modulator of TRPV1 receptor function of the present invention encompasses an antagonist and an agonist of the TRPV1 receptor and is useful for the treatment and/or prophylaxis of chronic pain such as neuropathic pain, inflammatory pain, nociceptive pain and the like; acute pain; surgical pain; pain of chronic rheumatoid arthritis or osteoarthritis; headache; migraine; pruritus; psoriasis; incontinence of urine; interstitial cystitis; overactive bladder; bronchial asthma; chronic coughing and the like. Preferably, it is prescribed as an analgesic for oral or parenteral administration.

For oral administration, the present modulator may be used in any conventional formulation of a solid formulation such as tablets, powders, granules and capsules; an aqueous formulation; an oily suspension; a liquid formulation such as syrup, elixir and the like. For parenteral administration, the modulator may be used as an aqueous or oily suspension for injection or a nasal solution. When these formulations are prepared, any conventional excipients, binders, lubricants, aqueous diluents, oily diluents, emulsifiers, suspension agents, preservatives and/or stabilizers etc. can be used.

The formulation of the present invention is prepared by incorporating(mixing, for example) a pharmaceutically acceptable excipient or diluent with the therapeutically effective amount of the compound, and in such cases the formulation is prepared by a known method using a well known, easily available components.

When the pharmaceutical composition of the present invention is prepared, the active ingredient is mixed or diluted with a carrier, or contained in a capsule, sachet, paper, or other carrier with a form of reservoir. The carrier is material of solid, semi-solid or liquid as a medium when it is used as a diluent, and tablets, pills, powders, buccals, elixirs, suspensions, emulsions, solutions, syrups, aerosols, and ointments are formulated using them comprising up to 10% of the active ingredient.

Any suitable carrier known to those skilled in the art may be used to prepare the formulation. In such formulation, the carrier is solid, liquid or a mixture of solid and liquid. For example, a compound of active ingredient is dissolved in a 4% dextrose/0.5% sodium citrate aquous solution to prepare an intravenous injection product. Solid formulations include powders, tablets and capsules.

Solid carrier is one or more material(s) useful as a flavor, a lubricant, a solubilizer, a suspension agent, a binder, a disintegrating agent and a material of capsules. Tablets for oral administration include a disintegrating agent such as corn starch and alginic acid, and/or a binder such as gelatin and acacia; a lubricant such as magnesium stearate, stearic acid, talc; and a suitable excipient such as calcium carbonate, sodium carbonate, lactose, calcium phosphate.

In a case of a powder, a solid carrier is finely ground to be mixed with a finely-ground active ingredient. In a case of a tablet, an active ingredient is mixed with a carrier having a necessary binding characteristics with a suitable ratio, and solidified in a desired shape and size. Both of the powder and the tablet may comprise about 1 to 99 volume % of the novel compound of the present invention as an active ingredient. Examples of the solid carrier include magnesium carbonate, magnesium stearate, talc, sugar, lactose, pectin, dextrin, starch, gelatin, tragacanth gum, methylcellulose, sodium carboxymethylcellulose, low melting point wax, and cocoa butter.

A liquid formulation includes a form of suspension, emulsion, syrup, elixir and the like. An active ingredient may be dissolved or suspended in a pharmaceutically acceptable carrier such as sterile water or an organic solvent or a mixture thereof. The active ingredient may also be dissolved in a suitable organic solvent such as an aqueous solution of propylene glycol. Other compositions may also be prepared by dispersing the finely-ground active ingredient in aqueous starch, a sodium carboxymethylcellulose solution or a suitable oil.

The dose of the compound of the present invention may vary in accordance with the administration routes, and the ages, weights and conditions of the patients, and a kind of the disease. When administered in an oral preparation, an amount of about 0.1 mg to 7,000 mg/day, preferably about 0.5 mg to 2,000 mg/day is usually administered to an adult in divided doses if necessary. When administered in an injection preparation, an amount of about 0.1 mg to 1,000 mg/day, preferably about 0.5 mg to 500 mg/day is usually administered to an adult.

### BEST MODE FOR CARRYING OUT THE INVENTION

A preferable embodiment of the present invention is the compound of the formula

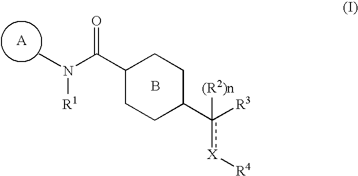

wherein R¹ is hydrogen or methyl,
and the combination of

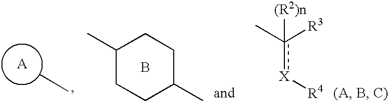

is selected from the group shown below;

TABLE 1

| | 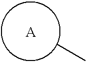 |
|---|---|
| A1 | 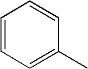 |
| A2 | 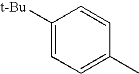 |
| A3 | 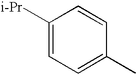 |
| A4 | 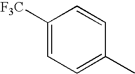 |
| A5 | 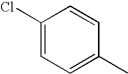 |
| A6 | 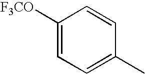 |
| A7 | 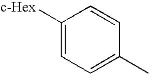 |
| A8 | 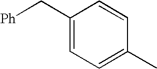 |
| A9 | 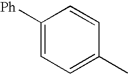 |
| A10 | 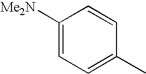 |
| A11 | 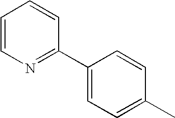 |
| A12 | 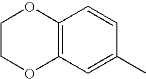 |
| A13 | 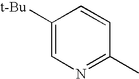 |
| A14 | 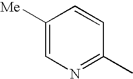 |
| A15 | 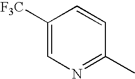 |
| A16 | 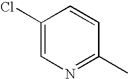 |
| A17 | 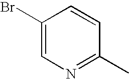 |
| A18 | 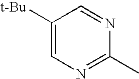 |
| A19 | 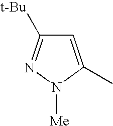 |
| A20 | 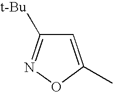 |
| A21 | 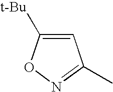 |
| A22 | 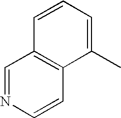 |
| A23 | 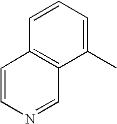 |
| A24 | 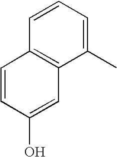 |
| A25 | 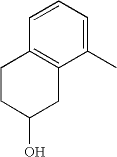 |
| A26 | 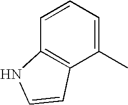 |
| A27 | 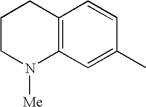 |

TABLE 2

| | 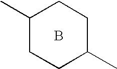 |
|---|---|
| B1 | 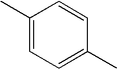 |
| B2 | 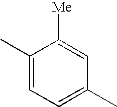 |
| B3 | 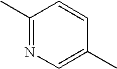 |
| B4 | 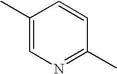 |
| B5 | 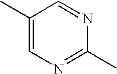 |
| B6 | 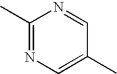 |

TABLE 3

| | 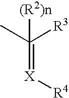 |
|---|---|
| C1 | 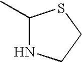 |
| C2 | 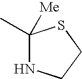 |
| C3 | 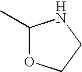 |
| C4 | 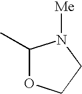 |
| C5 | 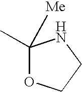 |
| C6 | 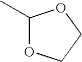 |
| C7 | 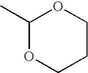 |
| C8 | 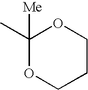 |
| C9 | 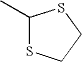 |
| C10 | 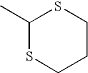 |
| C11 | 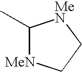 |
| C12 | 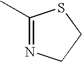 |
| C13 | 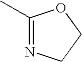 |
| C14 | 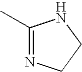 |
| C15 | 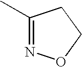 |
| C16 | 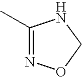 |
| C17 | 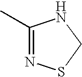 |
| C18 | 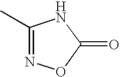 |
| C19 | 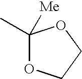 |
| C20 | 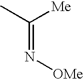 |
| C21 | 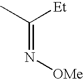 |

(A,B,C)=(A1,B1,C1),(A1,B1,C2),(A1,B1,C3),(A1,B1,C4),(A1,B1, C5),(A1,B1,C6),(A1,B1,C7),(A1,B1,C8),(A1,B1,C9),(A1,B1,C10), (A1,B1,C11),(A1,B1,C12),(A1,B1,C13),(A1,B1,C14),(A1,B1,C15), (A1,B1,C16),(A1,B1,C17),(A1,B1,C18),(A1,B1,C19),(A1,B1,C20), (A1,B1,C21),(A1,B2,C1),(A1,B2,C2),(A1,B2,C3),(A1,B2,C4), (A1,B2,C5),(A1,B2,C6),(A1,B2,C7),(A1,B2,C8),(A1,B2,C9),(A1, B2,C10),(A1,B2,C11),(A1,B2,C12),(A1,B2,C13),(A1,B2,C14),(A1, B2,C15),(A1,B2,C16),(A1,B2,C17),(A1,B2,C18),(A1,B2,C19),(A 1,B2,C20),(A1,B2,C21),(A1,B3,C1),(A1,B3,C2),(A1,B3,C3),(A1, B3,C4),(A1,B3,C5),(A1,B3,C6),(A1,B3,C7),(A1,B3,C8),(A1,B3,C 9),(A1,B3,C10),(A1,B3,C11),(A1,B3,C12),(A1,B3,C13),(A1,B3,C 14),(A1,B3,C15),(A1,B3,C16),(A1,B3,C17),(A1,B3,C18),(A1,B3, C19),(A1,B3,C20),(A1,B3,C21),(A1,B4,C1),(A1,B4,C2),(A1,B4,C 3),(A1,B4,C4),(A1,B4,C5),(A1,B4,C6),(A1,B4,C7),(A1,B4,C8),(A1,B4,C9),(A1,B4,C10),(A1,B4,C11),(A1,B4,C12),(A1,B4,C13),(A1,B4,C14),(A1,B4,C15),(A1,B4,C16),(A1,B4,C17),(A1,B4,C18), (A1,B4,C19),(A1,B4,C20),(A1,B4,C21),(A1,B5,C1),(A1,B5,C2),(A1,B5,C3),(A1,B5,C4),(A1,B5,C5),(A1,B5,C6),(A1,B5,C7),(A1,B 5,C8),(A1,B5,C9),(A1,B5,C10),(A1,B5,C11),(A1,B5,C12),(A1,B5, C13),(A1,B5,C14),(A1,B5,C15),(A1,B5,C16),(A1,B5,C17),(A1,B 5,C18),(A1,B5,C19),
(A1,B5,C20),(A1,B5,C21),(A1,B6,C1),(A1,B6,C2),(A1,B6,C3),(A 1,B6,C4),(A1,B6,C5),(A1,B6,C6),(A1,B6,C7),(A1,B6,C8),(A1,B6, C9),(A1,B6,C10),(A1,B6,C11),(A1,B6,C12),(A1,B6,C13),(A1,B6, C14),(A1,B6,C15),(A1,B6,C16),(A1,B6,C17),(A1,B6,C18),(A1,B 6,C19),(A1,B6,C20),(A1,B6,C21),(A2,B1,C1),(A2,B1,C2),(A2,B1, C3),(A2,B1,C4),(A2,B1,C5),(A2,B1,C6),(A2,B1,C7),(A2,B1,C8), (A2,B1,C9),(A2,B1,C10),(A2,B1,C11),(A2,B1,C12),(A2,B1,C13), (A2,B1,C14),(A2,B1,C15),(A2,B1,C16),(A2,B1,C17),(A2,B1,C18), (A2,B1,C19),(A2,B1,C20),(A2,B1, C21),(A2,B2,C1),(A2,B2,C2), (A2,B2,C3),(A2,B2,C4),(A2,B2,C5),(A2,B2,C6),(A2,B2,C7),(A2, B2,C8),(A2,B2,C9),(A2,B2,C10),(A2,B2,C11),(A2,B2,C12),(A2, B2,C13),(A2,B2,C14),(A2,B2,C15),(A2,B2,C16),(A2,B2,C17),(A2, B2,C18),(A2,B2,C19),(A2,B2,C20),(A2,B2,C21),(A2,B3,C1),(A2, B3,C2),(A2,B3,C3),(A2,B3,C4),(A2,B3,C5),(A2,B3,C6),(A2,B3, C7),(A2,B3,C8),(A2,B3,C9),(A2,B3,C10),(A2,B3,C11),(A2,B3,C12), (A2,B3,C13),(A2,B3,C14),(A2,B3,C15),(A2,B3,C16),(A2,B3,C 17),(A2,B3,C18),(A2,B3,C19),(A2,B3,C20),(A2,B3,C21),(A2,B4, C1),(A2,B4,C2),(A2,B4,C3),(A2,B4,C4),
(A2,B4,C5),(A2,B4,C6),(A2,B4,C7),(A2,B4,C8),(A2,B4,C9),(A2, B4,C10),(A2,B4,C11),(A2,B4,C12),(A2,B4,C13),(A2,B4,C14),(A2, B4,C15),(A2,B4,C16),(A2,B4,C17),(A2,B4,C18),(A2,B4,C19),(A 2,B4,C20),(A2,B4,C21),(A2,B5,C1),(A2,B5,C2),(A2,B5,C3),(A2, B5,C4),(A2,B5,C5),(A2,B5,C6),(A2,B5,C7),(A2,B5,C8),(A2,B5,C 9),(A2,B5,C10),(A2,B5,C11),(A2,B5,C12),(A2,B5,C13),(A2,B5,C 14),(A2,B5,C15),(A2,B5,C16),(A2,B5,C17),(A2,B5,C18),(A2,B5, C19),(A2,B5,C20),(A2,B5,C21),(A2,B6,C1),(A2,B6,C2),(A2,B6,C 3),(A2,B6,C4),(A2,B6,C5),(A2,B6,C6),(A2,B6,C7),(A2,B6,C8),(A2,B6,C9),(A2,B6,C10),(A2,B6,C11),(A2,B6,C12),(A2,B6,C13),(A2,B6,C14),(A2,B6,C15),(A2,B6,C16),(A2,B6,C17),(A2,B6,C18), (A2,B6,C19),(A2,B6,C20),(A2,B6,C21),(A3,B1,C1),(A3,B1,C2),(A3,B1,C3),(A3,B1,C4),(A3,B1,C5),(A3,B1,C6),(A3,B1,C7),(A3,B 1,C8),(A3,B1,C9),(A3,B1,C10),(A3,B1,C11),(A3,B1,C12),(A3,B1, C13),(A3,B1,C14),(A3,B1,C15),(A3,B1,C16),(A3,B1,C17),
(A3,B1,C18),(A3,B1,C19),(A3,B1,C20),(A3,B1, C21),(A3,B2,C1), (A3,B2,C2),(A3,B2,C3),(A3,B2,C4),(A3,B2,C5),(A3,B2,C6),(A3, B2,C7),(A3,B2,C8),(A3,B2,C9),(A3,B2,C10),(A3,B2,C11),(A3,B2, C12),(A3,B2,C13),(A3,B2,C14),(A3,B2,C15),(A3,B2,C16),(A3,B 2,C17),(A3,B2,C18),(A3,B2,C19),(A3,B2,C20),(A3,B2,C21),(A3, B3,C1),(A3,B3,C2),(A3,B3,C3),(A3,B3,C4),(A3,B3,C5),(A3,B3,C 6),(A3,B3,C7),(A3,B3,C8),(A3,B3,C9),(A3,B3,C10),(A3,B3,C11) (A3,B3,C12),(A3,B3,C13),(A3,B3,C14),(A3,B3,C15),(A3,B3,C16), (A3,B3,C17),(A3,B3,C18),(A3,B3,C19),(A3,B3,C20),(A3,B3,C21), (A3,B4,C1),(A3,B4,C2),(A3,B4,C3),(A3,B4,C4),(A3,B4,C5),(A3,B4,C6),(A3,B4,C7),(A3,B4,C8),(A3,B4,C9),(A3,B4,C10),(A3, B4,C11),(A3,B4,C12),(A3,B4,C13),(A3,B4,C14),(A3,B4,C15),(A3, B4,C16),(A3,B4,C17),(A3,B4,C18),(A3,B4,C19),(A3,B4,C20),(A 3,B4,C21),(A3,B5,C1),(A3,B5,C2),(A3,B5,C3),(A3,B5,C4),(A3,B 5,C5),(A3,B5,C6),(A3,B5,C7),(A3,B5,C8),(A3,B5,C9),(A3,B5,C10), (A3,B5,C11),(A3,B5,C12),(A3,B5,C13),(A3,B5,C14),(A3,B5,C 15),(A3,B5,C16),
(A3,B5,C17),(A3,B5,C18),(A3,B5,C19),(A3,B5,C20),(A3,B5,C21), (A3,B6,C1),(A3,B6,C2),(A3,B6,C3),(A3,B6,C4),(A3,B6,C5),(A3, B6,C6),(A3,B6,C7),(A3,B6,C8),(A3,B6,C9),(A3,B6,C10),(A3,B6, C11),(A3,B6,C12),(A3,B6,C13),(A3,B6,C14),(A3,B6,C15),(A3,B 6,C16),(A3,B6,C17),(A3,B6,C18),(A3,B6,C19),(A3,B6,C20),(A3, B6,C21),(A4,B1,C1),(A4,B1,C2),(A4,B1,C3),(A4,B1,C4),(A4,B1, C5),(A4,B1,C6),(A4,B1,C7),(A4,B1,C8),(A4,B1,C9),(A4,B1,C10), (A4,B1,C11),(A4,B1,C12),(A4,B1,C13),(A4,B1,C14),(A4,B1,C15), (A4,B1,C16),(A4,B1,C17),(A4,B1,C18),(A4,B1,C19),(A4,B1,C20), (A4,B1, C21),(A4,B2,C1),(A4,B2,C2),(A4,B2,C3),(A4,B2,C4), (A4,B2,C5),(A4,B2,C6),(A4,B2,C7),(A4,B2,C8),(A4,B2,C9),(A4, B2,C10),(A4,B2,C11),(A4,B2,C12),(A4,B2,C13),(A4,B2,C14),(A4, B2,C15),(A4,B2,C16),(A4,B2,C17),(A4,B2,C18),(A4,B2,C19),(A 4,B2,C20),(A4,B2,C21),(A4,B3,C1),
(A4,B3,C2),(A4,B3,C3),(A4,B3,C4),(A4,B3,C5),(A4,B3,C6),(A4, B3,C7),(A4,B3,C8),(A4,B3,C9),(A4,B3,C10),(A4,B3,C11),(A4,B3, C12),(A4,B3,C13),(A4,B3,C14),(A4,B3,C15),(A4,B3,C16),(A4,B 3,C17),(A4,B3,C18),(A4,B3,C19),(A4,B3,C20),(A4,B3,C21),(A4, B4,C1),(A4,B4,C2),(A4,B4,C3),(A4,B4,C4),(A4,B4,C5),(A4,B4,C 6),(A4,B4,C7),(A4,B4,C8),(A4,B4,C9),(A4,B4,C10),(A4,B4,C11) (A4,B4,C12),(A4,B4,C13),(A4,B4,C14),(A4,B4,C15),(A4,B4,C16), (A4,B4,C17),(A4,B4,C18),(A4,B4,C19),(A4,B4,C20),(A4,B4,C21), (A4,B5,C1),(A4,B5,C2),(A4,B5,C3),(A4,B5,C4),(A4,B5,C5),(A4,B5,C6),(A4,B5,C7),(A4,B5,C8),(A4,B5,C9),(A4,B5,C10),(A4, B5,C11),(A4,B5,C12),(A4,B5,C13),(A4,B5,C14),(A4,B5,C15),(A4, B5,C16),(A4,B5,C17),(A4,B5,C18),(A4,B5,C19),(A4,B5,C20),(A 4,B5,C21),(A4,B6,C1),(A4,B6,C2),(A4,B6,C3),(A4,B6,C4),(A4,B 6,C5),(A4,B6,C6),(A4,B6,C7),(A4,B6,C8),(A4,B6,C9),(A4,B6,C10), (A4,B6,C11),(A4,B6,C12),(A4,B6,C13),(A4,B6,C14),(A4,B6,C 15),(A4,B6,C16),(A4,B6,C17),(A4,B6,C18),(A4,B6,C19),(A4,B6, C20),(A4,B6,C21),(A5,B1,C1),(A5,B1,C2),(A5,B1,C3),(A5,B1,C4), (A5,B1,C5),(A5,B1,C6),(A5,B1,C7),(A5,B1,C8),(A5,B1,C9),(A 5, B1,C10)(A5, B1,C11),(A5,B1,C12),(A5,B1,C13),(A5,B1,C14),
(A5,B1,C15),(A5,B1,C16),(A5,B1,C17),(A5,B1,C18),(A5,B1,C19), (A5,B1,C20),(A5,B1, C21),(A5,B2,C1),(A5,B2,C2),(A5,B2,C3),(A5,B2,C4),(A5,B2,C5),(A5,B2,C6),(A5,B2,C7),(A5,B2,C8),(A5,B 2,C9),(A5,B2,C10),(A5,B2,C11),(A5,B2,C12),(A5,B2,C13),(A5,B 2,C14),(A5,B2,C15),(A5,B2,C16),(A5,B2,C17),(A5,B2,C18),(A5, B2,C19),(A5,B2,C20),(A5,B2,C21),(A5,B3.,C1),(A5,B3,C2),(A5,B 3,C3),(A5,B3,C4),(A5,B3,C5),(A5,B3,C6),(A5,B3,C7),(A5,B3,C8), (A5,B3,C9),(A5,B3,C10),(A5,B3,C11),(A5,B3,C12),(A5,B3,C13), (A5,B3,C14),(A5,B3,C15),(A5,B3,C16),(A5,B3,C17),(A5,B3,C18), (A5,B3,C19),(A5,B3,C20),(A5,B3,C21),(A5,B4,C1),(A5,B4,C2), (A5,B4,C3),(A5,B4,C4),(A5,B4,C5),(A5,B4,C6),(A5,B4,C7),(A 5,B4,C8),(A5,B4,C9),(A5,B4,C10),(A5,B4,C11),(A5,B4,C12),(A5, B4,C13),(A5,B4,C14),(A5,B4,C15),(A5,B4,C16),(A5,B4,C17),(A 5,B4,C18),(A5,B4,C19),(A5,B4,C20),(A5,B4,C21),(A5,B5,C1),(A 5,B5,C2),(A5,B5,C3),(A5,B5,C4),(A5,B5,C5),(A5,B5,C6),(A5,B5, C7),(A5,B5,C8),(A5,B5,C9),(A5,B5,C10),(A5,B5,C11),(A5,B5,C 12),(A5,B5,C13),
(A5,B5,C14),(A5,B5,C15),(A5,B5,C16),(A5,B5,C17),(A5,B5,C18), (A5,B5,C19),(A5,B5,C20),(A5,B5,C21),(A5,B6,C1),(A5,B6,C2), (A5, B6, C3),(A5, B6, C4),(A5, B6, C5),(A5, B6,C6),(A5, B6, C7),(A5, B6, C8),(A5, B6,C9),(A5, B6, C10),(A5, B6,C11),(A5, B6, C12),(A5, B 6,C13),(A5,B6,C14),(A5,B6,C15),(A5,B6,C16),(A5,B6,C17),(A6, B6,C18),(A5,B6,C19),(A5,B6,C20),(A5,B6,C21),(A6,B1,C1), (A6, B1,C2),(A6,B1,C3),(A6,B1,C4),(A6,B1,C5),(A6,B1,C6),(A6,B1,C 7),(A6,B1,C8),(A6,B1,C9),(A6,B1,C10),(A6,B1,C11),(A6,B1,C12), (A6,B1,C13),(A6,B1,C14),(A6,B1,C15),(A6,B1,C16),(A6,B1,C17), (A6,B1,C18),(A6,B1,C19),(A6,B1,C20),(A6,B1, C21),(A6,B2,C 1),(A6,B2,C2),(A6,B2,C3),(A6,B2,C4),(A6,B2,C5),(A6,B2,C6),(A6,B2,C7),(A6,B2,C8),(A6,B2,C9),(A6,B2,C10),(A6,B2,C11),(A6, B2,C12),(A6,B2,C13),(A6,B2,C14),(A6,B2,C15),(A6,B2,C16),(A 6,B2,C17),(A6,B2,C18),(A6,B2,C19),(A6,B2,C20),(A6,B2,C21),(A 6,B3,C1),(A6,B3,C2),(A6,B3,C3),(A6,B3,C4),(A6,B3,C5),
(A6,B3,C6),(A6,B3,C7),(A6,B3,C8),(A6,B3,C9),(A6,B3,C10),(A6, B3,C11),(A6,B3,C12),(A6,B3,C13),(A6,B3,C14),(A6,B3,C15),(A 6,B3,C16),(A6,B3,C17),(A6,B3,C18),(A6,B3,C19),(A6,B3,C20),(A 6,B3,C21),(A6,B4,C1),(A6,B4,C2),(A6,B4,C3),(A6,B4,C4),(A6, B4,C5),(A6,B4,C6),(A6,B4,C7),(A6,B4,C8),(A6,B4,C9),(A6,B4,C 10),(A6,B4,C11),(A6,B4,C12),(A6,B4,C13),(A6,B4,C14),(A6,B4, C15),(A6,B4,C16),(A6,B4,C17),(A6,B4,C18),(A6,B4,C19),(A6,B4, C20),(A6,B4,C21),(A6,B5,C1),(A6,B5,C2),(A6,B5,C3),(A6,B5,C 4),(A6,B5,C5),(A6,B5,C6),(A6,B5,C7),(A6,B5,C8),(A6,B5,C9),(A 6,B5,C10),(A6,B5,C11),(A6,B5,C12),(A6,B5,C13),(A6,B5,C14), (A6,B5,C15),(A6,B5,C16),(A6,B5,C17),(A6,B5,C18),(A6,B5,C19), (A6,B5,C20),(A6,B5,C21),(A6,B6,C1),(A6,B6,C2),(A6,B6,C3), A6,B6,C4),(A6,B6,C5),(A6,B6,C6),(A6,B6,C7),(A6,B6,C8),(A6,B 6,C9),(A6,B6,C10),(A6,B6,C11),(A6,B6,C12),(A6,B6,C13),(A6,B 6,C14),(A6,B6,C15),(A6,B6,C16),(A6,B6,C17),(A6,B6,C18),(A6, B6,C19),(A6,B6,C20),(A6,B6,C21),(A7,B1,C1),(A7,B1,C2),(A7,B 1,C3),(A7,B1,C4),(A7,B1,C5),(A7,B1,C6),(A7,B1,C7),(A7,B1,C8), (A7,B1,C9),(A7,B1,C10),(A7,B1,C11),
(A7,B1,C12),(A7,B1,C13),(A7,B1,C14),(A7,B1,C15),(A7,B1,C16) (A7,B1,C17),(A7,B1,C18),(A7,B1,C19),(A7,B1,C20),(A7,B1,C21), (A7,B2,C1),(A7,B2,C2),(A7,B2,C3),(A7,B2,C4),(A7,B2,C5),(A 7,B2,C6),(A7,B2,C7),(A7,B2,C8),(A7,B2,C9),(A7,B2,C10),(A7,B 2,C11),(A7,B2,C12),(A7,B2,C13),(A7,B2,C14),(A7,B2,C15),(A7, B2,C16),(A7,B2,C17),(A7,B2,C18),(A7,B2,C19),(A7,B2,C20),(A7, B2,C21),(A7,B3,C1),(A7,B3,C2),(A7,B3,C3),(A7,B3,C4),(A7,B3, C5),(A7,B3,C6),(A7,B3,C7),(A7,B3,C8),(A7,B3,C9),(A7,B3,C10), (A7,B3,C11),(A7,B3,C12),(A7,B3,C13),(A7,B3,C14),(A7,B3,C15), (A7,B3,C16),(A7,B3,C17),(A7,B3,C18),(A7,B3,C19),(A7,B3,C 20),(A7,B3,C21),(A7,B4,C1),(A7,B4,C2),(A7,B4,C3),(A7,B4,C4), (A7,B4,C5),(A7,B4,C6),(A7,B4,C7),(A7,B4,C8),(A7,B4,C9),(A7, B4,C10),(A7,B4,C11),(A7,B4,C12),(A7,B4,C13),(A7,B4,C14),(A 7,B4,C15),(A7,B4,C16),(A7,B4,C17),(A7,B4,C18),(A7,B4,C19),(A 7,B4,C20),(A7,B4,C21),(A7,B5,C1),(A7,B5,C2),(A7,B5,C3),(A7, B5,C4),(A7,B5,C5),(A7,B5,C6),(A7,B5,C7),(A7,B5,C8),(A7,B5, C9),(A7,B5,C10),
(A7,B5,C11),(A7,B5,C12),(A7,B5,C13),(A7,B5,C14),(A7,B5,C15), (A7,B5,C16),(A7,B5,C17),(A7,B5,C18),(A7,B5,C19),(A7,B5,C20), (A7,B5,C21),(A7,B6,C1),(A7,B6,C2),(A7,B6,C3),(A7,B6,C4),(A 7,B6,C5),(A7,B6,C6),(A7,B6,C7),(A7,B6,C8),(A7,B6,C9),(A7,B 6,C10),(A7,B6,C11),(A7,B6,C12),(A7,B6,C13),(A7,B6,C14),(A7, B6,C15),(A7,B6,C16),(A7,B6,C17),(A7,B6,C18),(A7,B6,C19),(A7, B6,C20),(A7,B6,C21),(A8,B1,C1),(A8,B1,C2),(A8,B1,C3),(A8,B 1,C4),(A8,B1,C5),(A8,B1,C6),(A8,B1,C7),(A8,B1,C8),(A8,B1,C9), (A8,B1,C10),(A8,B1,C11),(A8,B1,C12),(A8,B1,C13),(A8,B1,C14), (A8,B1,C15),(A8,B1,C16),(A8,B1,C17),(A8,B1,C18),(A8,B1,C 19),(A8,B1,C20),(A8,B1, C21),(A8,B2,C1),(A8,B2,C2),(A8,B2,C3), (A8,B2,C4),(A8,B2,C5),(A8,B2,C6),(A8,B2,C7),(A8,B2,C8),(A 8,B2,C9),(A8,B2,C10),(A8,B2,C11),(A8,B2,C12),(A8,B2,C13),(A 8,B2,C14),(A8,B2,C15),(A8,B2,C16),(A8,B2,C17),(A8,B2,C18),(A 8,B2,C19),(A8,B2,C20),(A8,B2,C21),(A8,B3,C1),(A8,B3,C2),(A 8,B3,C3),(A8,B3,C4),(A8,B3,C5),(A8,B3,C6),(A8,B3,C7),(A8,B3, C8),(A8,B3,C9),
(A8,B3,C10),(A8,B3,C11),(A8,B3,C12),(A8,B3,C13),(A8,B3,C14), (A8,B3,C15),(A8,B3,C16),(A8,B3,C17),(A8,B3,C18),(A8,B3,C19), (A8,B3,C20),(A8,B3,C21),(A8,B4,C1),(A8,B4,C2),(A8,B4,C3), (A8,B4,C4),(A8,B4,C5),(A8,B4,C6),(A8,B4,C7),(A8,B4,C8),(A8, B4,C9),(A8,B4,C10),(A8,B4,C11),(A8,B4,C12),(A8,B4,C13),(A8, B4,C14),(A8,B4,C15),(A8,B4,C16),(A8,B4,C17),(A8,B4,C18),(A8, B4,C19),(A8,B4,C20),(A8,B4,C21),(A8,B5,C1),(A8,B5,C2),(A8, B5,C3),(A8,B5,C4),(A8,B5,C5),(A8,B5,C6),(A8,B5,C7),(A8,B5,C 8),(A8,B5,C9),(A8,B5,C10),(A8,B5,C11),(A8,B5,C12),(A8,B5,C13), (A8,B5,C14),(A8,B5,C15),(A8,B5,C16),(A8,B5,C17),(A8,B5,C 18),(A8,B5,C19),(A8,B5,C20),(A8,B5,C21),(A8,B6,C1),(A8,B6,C 2),(A8,B6,C3),(A8,B6,C4),(A8,B6,C5),(A8,B6,C6),(A8,B6,C7),(A 8,B6,C8),(A8,B6,C9),(A8,B6,C10),(A8,B6,C11),(A8,B6,C12),(A 8,B6,C13),(A8,B6,C14),(A8,B6,C15),(A8,B6,C16),(A8,B6,C17),(A 8,B6,C18),(A8,B6,C19),(A8,B6,C20),(A8,B6,C21),(A9,B1,C1),(A 9,B1,C2),(A9,B1,C3),(A9,B1,C4),(A9,B1,C5),(A9,B1,C6),(A9,B 1,C7),(A9,B1,C8),(A9,B1,C9),(A9,B1,C10),(A9,B1,C11),(A9,B1, C12),(A9,B1,C13),(A9,B1,C14),(A9,B1,C15),(A9,B1,C16),(A9,B1, C17),(A9,B1,C18),(A9,B1,C19),(A9,B1,C20),(A9,B1,C21),(A9,B 2,C1),(A9,B2,C2),(A9,B2,C3),(A9,B2,C4),(A9,B2,C5),(A9,B2,C6), (A9,B2,C7),(A9,B2,C8),(A9,B2,C9),(A9,B2,C10),(A9,B2,C11), (A9,B2,C12),(A9,B2,C13),(A9,B2,C14),(A9,B2,C15),(A9,B2,C16), (A9,B2,C17),(A9,B2,C18),(A9,B2,C19),(A9,B2,C20),(A9,B2,C21),
(A9,B3,C1),(A9,B3,C2),(A9,B3,C3),(A9,B3,C4),(A9,B3,C5),(A9, B3,C6),(A9,B3,C7),(A9,B3,C8),(A9,B3,C9),(A9,B3,C10),(A9,B3, C11),(A9,B3,C12),(A9,B3,C13),(A9,B3,C14),(A9,B3,C15),(A9,B3, C16),(A9,B 3,C17),(A9,B3,C18),(A9,B3,C19),(A9,B3,C20),(A9,B 3,C21),(A9,B4,C1),(A9,B4,C2),(A9,B4,C3),(A9,B4,C4),(A9,B4,C 5),(A9,B4,C6),(A9,B4,C7),(A9,B4,C8),(A9,B4,C9),(A9,B4,C10), (A9,B4,C11),(A9,B4,C12),(A9,B4,C13),(A9,B4,C14),(A9,B4,C15), (A9,B4,C16),(A9,B4,C17),(A9,B4,C18),(A9,B4,C19),(A9,B4,C20), (A9,B4,C21),(A9,B5,C1),(A9,B5,C2),(A9,B5,C3),(A9,B5,C4),(A 9,B5,C5),(A9,B5,C6),(A9,B5,C7),(A9,B5,C8),(A9,B5,C9),(A9,B 5,C10),(A9,B5,C11),(A9,B5,C12),(A9,B5,C13),(A9,B5,C14),(A9, B5,C15),(A9,B5,C16),(A9,B5,C17),(A9,B5,C18),(A9,B5,C19),(A9, B5,C20),(A9,B5,C21),(A9,B6,C1),(A9,B6,C2),(A9,B6,C3),(A9,B 6,C4),(A9,B6,C5),(A9,B6,C6),(A9,B6,C7),(A9,B6,C8),(A9,B6,C9), (A9,B6,C10),(A9,B6,C11),(A9,B6,C12),(A9,B6,C13),(A9,B6,C14), (A9,B6,C15),(A9,B6,C16),(A9,B6,C17),(A9,B6,C18),(A9,B6,C 19),(A9,B6,C20),(A9,B6,C21),(A10,B1,C1),(A10,B1,C2),(A10,B1, C3),(A10,B1,C4),(A10,B1,C5),(A10,B1,C6),(A10,B1,C7),(A10,B 1,C8),(A10,B1,C9),(A10,B1,C10),(A10,B1,C11),(A10,B1,C12),
(A10,B1,C13),(A10,B1,C14),(A10,B1,C15),(A10,B1,C16),(A10,B1, C17),(A10,B1,C18),(A10,B1,C19),(A10,B1,C20),(A10,B1,C21),(A 10,B2,C1),(A10,B2,C2),(A10,B2,C3),(A10,B2,C4),(A10,B2,C5), (A10,B2,C6),(A10,B2,C7),(A10,B2,C8),(A10,B2,C9),(A10,B2,C10), (A10,B2,C11),(A10,B2,C12),(A10,B2,C13),(A10,B2,C14),(A10, B2,C15),(A10,B2,C16),(A10,B2,C17),(A10,B2,C18),(A10,B2,C19), (A10,B2,C20),(A10,B2,C21),(A10,B3,C1),(A10,B3,C2),(A10,B3, C3),(A10,B3,C4),(A10,B3,C5),(A10,B3,C6),(A10,B3,C7),(A10,B3, C8),(A10,B3,C9),(A10,B3,C10),(A10,B3,C11),(A10,B3,C12),(A1 0,B3,C13),(A10,B3,C14),(A10,B3,C15),(A10,B3,C16),(A10,B3,C17), (A10,B3,C18),(A10,B3,C19),(A10,B3,C20),(A10,B3,C21),(A10, B4,C1),(A10,B4,C2),(A10,B4,C3),(A10,B4,C4),(A10,B4,C5),(A1 0,B4,C6),(A10,B4,C7),(A10,B4,C8),(A10,B4,C9),(A10,B4,C10), A10,B4,C11),(A10,B4,C12),(A10,B4,C13),(A10,B4,C14),(A10,B4, C15),(A10,B4,C16),(A10,B4,C17),(A10,B4,C18),(A10,B4,C19),(A 10,B4,C20),(A10,B4,C21),(A10,B5,C1),(A10,B5,C2),(A10,B5,C3), (A10,B5,C4),(A10,B5,C5),(A10,B5,C6),(A10,B5,C7),(A10,B5,C8), (A10,B5,C9),(A10,B5,C10),(A10,B5,C11),(A10,B5,C12),(A10,B 5,C13),(A10,B5,C14),(A10,B5,C15),(A10,B5,C16),(A10,B5,C17), (A10,B5,C18),(A10,B5,C19),(A10,B5,C20),(A10,B5,C21),(A10,B6, C1),(A10,B6,C2),(A10,B6,C3),(A10,B6,C4),(A10,B6,C5),(A10,B 6,C6),(A10,B6,C7),(A10,B6,C8),(A10,B6,C9),(A10,B6,C10),(A10, B6,C11),(A10,B6,C12),(A10,B6,C13),(A10,B6,C14),(A10,B6,C15),
(A10,B6,C16),(A10,B6,C17),(A10,B6,C18),(A10,B6,C19),(A10,B6, C20),(A10,B6,C21),(A11,B1,C1),(A11,B1,C2),(A11,B1,C3),(A11, B1,C4),(A11,B1,C5),(A11,B1,C6),(A11,B1,C7),(A11,B1,C8),(A1 1,B1,C9),(A11,B1,C10),(A11,B1,C11),(A11,B1,C12),(A11,B1,C13), (A11,B1,C14),(A11,B1,C15),(A11,B1,C16),(A11,B1,C17),(All, B1,C18),(A11,B1,C19),(A11,B1,C20),(A11,B1, C21),(A11,B2,C1), (A11,B2,C2),(A11,B2,C3),(A11,B2,C4),(A11,B2,C5),(A11,B2,C6), (A11,B2,C7),(A11,B2,C8),(A11,B2,C9),(A11,B2,C10),(A11,B2,C 11),(A11,B2,C12),(A11,B2,C13),(A11,B2,C14),(A11,B2,C15),(A11, B2,C16),(A11,B2,C17),(A11,B2,C18),(A11,B2,C19),(A11,B2,C20), (A11,B2,C21),(A11,B3,C1),(A11,B3,C2),(A11,B3,C3),(A11,B3, C4),(A11,B3,C5),(A11,B3,C6),(A11,B3,C7),(A11,B3,C8),(A11,B 3,C9),(A11,B3,C10),(A11,B3,C11),(A11,B3,C12),(A11,B3,C13), A11,B3,C14),(A11,B3,C15),(A11,B3,C16),(A11,B3,C17),(A11,B3, C18),(A11,B3,C19),(A11,B3,C20),(A11,B3,C21),(A11,B4,C1),(A1 1,B4,C2),(A11,B4,C3),(A11,B4,C4),(A11,B4,C5),(A11,B4,C6),(A 11,B4,C7),(A11,B4,C8),(A11,B4,C9),(A11,B4,C10),(A11,B4,C11), (A11,B4,C12),(A11,B4,C13),(A11,B4,C14),(A11,B4,C15),(A11,B 4,C16),(A11,B4,C17),(A11,B4,C18),(A11,B4,C19),(A11,B4,C20), (A11,B4,C21),(A11,B5,C1),(A11,B5,C2),(A11,B5,C3),(A11,B5,C4), (A11,B5,C5),(A11,B5,C6),(A11,B5,C7),(A11,B5,C8),(A11,B5,C 9),(A11,B5,C10),(A11,B5,C11),(A11,B5,C12),(A11,B5,C13),(A11, B5,C14),(A11,B5,C15),(A11,B5,C16),(A11,B5,C17),(A11,B5,C18),
(A11,B5,C19),(A11,B5,C20),(A11,B5,C21),(A11,B6,C1),(A11,B6, C2),(A11,B6,C3),(A11,B6,C4),(A11,B6,C5),(A11,B6,C6),(A11,B6, C7),(A11,B6,C8),(A11,B6,C9),(A11,B6,C10),(A11,B6,C11),(A11, B6,C12),(A11,B6,C13),(A11,B6,C14),(A11,B6,C15),(A11,B6,C16), (A11,B6,C17),(A11,B6,C18),(A11,B6,C19),(A11,B6,C20),(All, B6,C21),(A12,B1,C1),(A12,B1,C2),(A12,B1,C3),(A12,B1,C4),(A1 2,B1,C5),(A12,B1,C6),(A12,B1,C7),(A12,B1,C8),(A12,B1,C9),(A 12,B1,C10),(A12,B1,C11),(A12,B1,C12),(A12,B1,C13),(A12,B1,C 14),(A12,B1,C15),(A12,B1,C16),(A12,B1,C17),(A12,B1,C18),(A1 2,B1,C19),(A12,B1,C20),(A12,B1, C21),(A12,B2,C1),(A12,B2,C2), (A12,B2,C3),(A12,B2,C4),(A12,B2,C5),(A12,B2,C6),(A12,B2,C7), (A12,B2,C8),(A12,B2,C9),(A12,B2,C10),(A12,B2,C11),(A12,B2, C12),(A12,B2,C13),(A12,B2,C14),(A12,B2,C15),(A12,B2,C16),(A12,B2,C17),(A12,B2,C18),(A12,B2,C19),(A12,B2,C20),(A12,B2, C21),(A12,B3,C1),(A12,B3,C2),(A12,B3,C3),(A12,B3,C4),(A12,B 3,C5),(A12,B3,C6),(A12,B3,C7),(A12,B3,C8),(A12,B3,C9),(A12, B3,C10),(A12,B3,C11),(A12,B3,C12),(A12,B3,C13),(A12,B3,C14), (A12,B3,C15),(A12,B3,C16),(A12,B3,C17),(A12,B3,C18),(A12,B 3,C19),(A12,B3,C20),(A12,B3,C21),(A12,B4,C1),(A12,B4,C2),(A 12,B4,C3),(A12,B4,C4),(A12,B4,C5),(A12,B4,C6),(A12,B4,C7),(A 12,B4,C8),(A12,B4,C9),(A12,B4,C10),(A12,B4,C11),(A12,B4,C12), (A12,B4,C13),(A12,B4,C14),(A12,B4,C15),(A12,B4,C16),(A12, B4,C17),(A12,B4,C18),(A12,B4,C19),(A12,B4,C20),(A12,B4,C21),
(A12,B5,C1),(A12,B5,C2),(A12,B5,C3),(A12,B5,C4),(A2,B5,C5), (A112,B5,C6),(A12,B5,C7),(A12,B5,C8),(A12,B5,C9),(A12,B5,C10), (A12,B5,C11),(A12,B5,C12),(A12,B5,C13),(A12,B5,C14),(A12, B5,C15),(A12,B5,C16),(A12,B5,C17),(A12,B5,C18),(A12,B5,C19), (A12,B5,C20),(A12,B5,C21),(A12,B6,C1),(A12,B6,C2),(A12,B6, C3),(A12,B6,C4),(A12,B6,C5),(A12,B6,C6),(A12,B6,C7),(A12,B 6,C8),(A12,B6,C9),(A12,B6,C10),(A12,B6,C11),(A12,B6,C12),(A 12,B6,C13),(A12,B6,C14),(A12,B6,C15),(A12,B6,C16),(A12,B6,C 17),(A12,B6,C18),(A12,B6,C19),(A12,B6,C20),(A12,B6,C21),(A1 3,B1,C1),(A13,B1,C2),(A13,B1,C3),(A13,B1,C4),(A13,B1,C5),(A 13,B1,C6),(A13,B1,C7),(A13,B1,C8),(A13,B1,C9),(A13,B1,C10), (A13,B1,C11),(A13,B1,C12),(A13,B1,C13),(A13,B1,C14),(A13,B1, C15),(A13,B1,C16),(A13,B1,C17),(A13,B1,C18),(A13,B1,C19),(A13,B1,C20),(A13,B1, C21),(A13,B2,C1),(A13,B2,C2),(A13,B2,C3), (A13,B2,C4),(A13,B2,C5),(A13,B2,C6),(A13,B2,C7),(A13,B2,C 8),(A13,B2,C9),(A13,B2,C10),(A13,B2,C11),(A13,B2,C12),(A13, B2,C13),(A13,B2,C14),(A13,B2,C15),(A13,B2,C16),(A13,B2,C17), (A13,B2,C18),(A13,B2,C19),(A13,B2,C20),(A13,B2,C21),(A13,B 3,C1),(A13,B3,C2),(A13,B3,C3),(A13,B3,C4),(A13,B3,C5),(A13, B3,C6),(A13,B3,C7),(A13,B3,C8),(A13,B3,C9),(A13,B3,C10),(A1 3,B3,C11),(A13,B3,C12),(A13,B3,C13),(A13,B3,C14),(A13,B3,C15), (A13,B3,C16),(A13,B3,C17),(A13,B3,C18),(A13,B3,C19),(A13, B3,C20),(A13,B3,C21),(A13,B4,C1),(A13,B4,C2),(A13,B4,C3),(A 13,B4,C4),(A13,B4,C5),(A13,B4,C6),(A13,B4,C7),(A13,B4,C8), (A13,B4,C9),(A13,B4,C10),(A13,B4,C11),(A13,B4,C12),(A13,B4, C13),(A13,B4,C14),(A13,B4,C15),(A13,B4,C16),
(A13,B4,C17),(A13,B4,C18),(A13,B4,C19),(A13,B4,C20),(A13,B4, C21),(A13,B5,C1),(A13,B5,C2),(A13,B5,C3),(A13,B5,C4),(A13, B5,C5),(A13,B5,C6),(A13,B5,C7),(A13,B5,C8),(A13,B5,C9),(A13, B5,C10),(A13,B5,C11),(A13,B5,C12),(A13,B5,C13),(A13,B5,C14), (A13,B5,C15),(A13,B5,C16),(A13,B5,C17),(A13,B5,C18),(A13, B5,C19),(A13,B5,C20),(A13,B5,C21),(A13,B6,C1),(A13,B6,C2),(A 13,B6,C3),(A13,B6,C4),(A13,B6,C5),(A13,B6,C6),(A13,B6,C7), (A13,B6,C8),(A13,B6,C9),(A13,B6,C10),(A13,B6,C11),(A13,B6,C, 12),(A13,B6,C13),(A13,B6,C14),(A13,B6,C15),(A13,B6,C16),(A1 3,B6,C17),(A13,B6,C18),(A13,B6,C19),(A13,B6,C20),(A13,B6,C21), (A14,B1,C1),(A14,B1,C2),(A14,B1,C3),(A14,B1,C4),(A14,B1, C5),(A14,B1,C6),(A14,B1,C7),(A14,B1,C8),(A14,B1,C9),(A14,B1, C10),(A14,B1,C11),(A14,B1,C12),(A14,B1,C13),(A14,B1,C14),(A14,B1,C15),(A14,B1,C16),(A14,B1,C17),(A14,B1,C18),(A14,B1, C19),(A14,B1,C20),(A14,B1, C21),(A14,B2,C1),(A14,B2,C2),(A14, B2,C3),(A14,B2,C4),(A14,B2,C5),(A14,B2,C6),(A14,B2,C7),(A1 4,B2,C8),(A14,B2,C9),(A14,B2,C10),(A14,B2,C11),(A14,B2,C12), (A14,B2,C13),(A14,B2,C14),(A14,B2,C15),(A14,B2,C16),(A14,B 2,C17),(A14,B2,C18),(A14,B2,C19),(A14,B2,C20),(A14,B2,C21), (A14,B3,C1),(A14,B3,C2),(A14,B3,C3),(A14,B3,C4),(A14,B3,C5), (A14,B3,C6),(A14,B3,C7),(A14,B3,C8),(A14,B3,C9),(A14,B3,C10), (A14,B3,C11),(A14,B3,C12),(A14,B3,C13),(A14,B3,C14),(A14, B3,C15),(A14,B3,C16),(A14,B3,C17),(A14,B3,C18),(A14,B3,C19),
(A14,B3,C20),(A14,B3,C21),(A14,B4,C1),(A14,B4,C2),(A14,B4,C 3),(A14,B4,C4),(A14,B4,C5),(A14,B4,C6),(A14,B4,C7),(A14,B4, C8),(A14,B4,C9),(A14,B4,C10),(A14,B4,C11),(A14,B4,C12),(A14, B4,C13),(A14,B4,C14),(A14,B4,C15),(A14,B4,C16),(A14,B4,C17), (A14,B4,C18),(A14,B4,C19),(A14,B4,C20),(A14,B4,C21),(A14, B5,C1),(A14,B5,C2),(A14,B5,C3),(A14,B5,C4),(A14,B5,C5),(A14, B5,C6),(A14,B5,C7),(A14,B5,C8),(A14,B5,C9),(A14,B5,C10),(A 14,B5,C11),(A14,B5,C12),(A14,B5,C13),(A14,B5,C14),(A14,B5,C 15),(A14,B5,C16),(A14,B5,C17),(A14,B5,C18),(A14,B5,C19),(A1 4,B5,C20),(A14,B5,C21),(A14,B6,C1),(A14,B6,C2),(A14,B6,C3), (A14,B6,C4),(A14,B6,C5),(A14,B6,C6),(A14,B6,C7),(A14,B6,C8), (A14,B6,C9),(A14,B6,C10),(A14,B6,C11),(A14,B6,C12),(A14,B6, C13),(A14,B6,C14),(A14,B6,C15),(A14,B6,C16),(A14,B6,C17),(A 14,B6,C18),(A14,B6,C19),(A14,B6,C20),(A14,B6,C21),(A15,B1, C1),(A15,B1,C2),(A15,B1,C3),(A15,B1,C4),(A15,B1,C5),(A15,B1, C6),(A15,B1,C7),(A15,B1,C8),(A15,B1,C9),(A15,B1,C10),(A15, B1,C11),(A15,B1,C12),(A15,B1,C13),(A15,B1,C14),(A15,B1,C15), (A15,B1,C16),(A15,B1,C17),(A15,B1,C18),(A15,B1,C19),(A15,B 1,C20),(A15,B1, C21),(A15,B2,C1),(A15,B2,C2),(A15,B2,C3),(A1 5,B2,C4),(A15,B2,C5),(A15,B2,C6),(A15,B2,C7),(A15,B2,C8),(A 15,B2,C9),(A15,B2,C10),(A15,B2,C11),(A15,B2,C12),(A15,B2,C13), (A15,B2,C14),(A15,B2,C15),(A15,B2,C16),(A15,B2,C17),(A15, B2,C18),(A15,B2,C19),(A15,B2,C20),(A15,B2,C21),(A15,B3,C1),
(A15,B3,C2),(A15,B3,C3),(A15,B3,C4),(A15,B3,C5),(A15,B3,C6), (A15,B3,C7),(A15,B3,C8),(A15,B3,C9),(A15,B3,C10),(A15,B3,C 11),(A15,B3,C12),(A15,B3,C13),(A15,B3,C14),(A15,B3,C15),(A1 5,B3,C16),(A15,B3,C17),(A15,B3,C18),(A15,B3,C19),(A15,B3,C2 0),(A15,B3,C21),(A15,B4,C1),(A15,B4,C2),(A15,B4,C3),(A15,B4, C4),(A15,B4,C5),(A15,B4,C6),(A15,B4,C7),(A15,B4,C8),(A15,B 4,C9),(A15,B4,C10),(A15,B4,C11),(A15,B4,C12),(A15,B4,C13),(A 15,B4,C14),(A15,B4,C15),(A15,B4,C16),(A15,B4,C17),(A15,B4, C18),(A15,B4,C19),(A15,B4,C20),(A15,B4,C21),(A15,B5,C1),(A1 5,B5,C2),(A15,B5,C3),(A15,B5,C4),(A15,B5,C5),(A15,B5,C6),(A 15,B5,C7),(A15,B5,C8),(A15,B5,C9),(A15,B5,C10),(A15,B5,C11), (A15,B5,C12),(A15,B5,C13),(A15,B5,C14),(A15,B5,C15),(A15,B 5,C16),(A15,B5,C17),(A15,B5,C18),(A15,B5,C19),(A15,B5,C20), (A15,B5,C21),(A15,B6,C1),(A15,B6,C2),(A15,B6,C3),(A15,B6,C4), (A15,B6,C5),(A15,B6,C6),(A15,B6,C7),(A15,B6,C8),(A15,B6,C 9),(A15,B6,C10),(A15,B6,C11),(A15,B6,C12),(A15,B6,C13),(A15, B6,C14),(A15,B6,C15),(A15,B6,C16),(A15,B6,C17),(A15,B6,C18), (A15,B6,C19),(A15,B6,C20),(A15,B6,C21),(A16,B1,C1),(A16,B 1,C2),(A16,B1,C3),(A16,B1,C4),(A16,B1,C5),(A16,B1,C6),(A16, B1,C7),(A16,B1,C8),(A16,B1,C9),(A16,B1,C10),(A16,B1,C11),(A 16,B1,C12),(A16,B1,C13),(A16,B1,C14),(A16,B1,C15),(A16,B1,C 16),(A16,B1,C17),(A16,B1,C18),(A16,B1,C19),(A16,B1,C20),(A1 6,B1, C21),(A16,B2,C1),(A16,B2,C2),(A16,B2,C3),(A16,B2,C4),(A 16,B2,C5),(A16,B2′,C6),(A16,B2,C7),(A16,B2,C8),(A16,B2,C9), (A16,B2,C10),(A16,B2,C11),(A16,B2,C12),(A16,B2,C13),(A16,B2, C14),(A16,B2,C15),(A16,B2,C16),(A16,B2,C17),
(A16,B2,C18),(A16,B2,C19),(A16,B2,C20),(A16,B2,C21),(A16,B3, C1),(A16,B3,C2),(A16,B3,C3),(A16,B3,C4),(A16,B3,C5),(A16,B 3,C6),(A16,B3,C7),(A16,B3,C8),(A16,B3,C9),(A16,B3,C10),(A16, B3,C11),(A16,B3,C12),(A16,B3,C13),(A16,B3,C14),(A16,B3,C15), (A16,B3,C16),(A16,B3,C17),(A16,B3,C18),(A16,B3,C19),(A16, B3,C20),(A16,B3,C21),(A16,B4,C1),(A16,B4,C2),(A16,B4,C3),(A 16,B4,C4),(A16,B4,C5),(A16,B4,C6),(A16,B4,C7),(A16,B4,C8),(A 16,B4,C9),(A16,B4,C10),(A16,B4,C11),(A16,B4,C12),(A16,B4,C 13),(A16,B4,C14),(A16,B4,C15),(A16,B4,C16),(A16,B4,C17),(A1 6,B4,C18),(A16,B4,C19),(A16,B4,C20),(A16,B4,C21),(A16,B5,C1), (A16,B5,C2),(A16,B5,C3),(A16,B5,C4),(A16,B5,C5),(A16,B5,C 6),(A16,B5,C7),(A16,B5,C8),(A16,B5,C9),(A16,B5,C10),(A16,B5, C11),(A16,B5,C12),(A16,B5,C13),(A16,B5,C14),(A16,B5,C15),(A 16,B5,C16),(A16,B5,C17),(A16,B5,C18),(A16,B5,C19),(A16,B5, C20),(A16,B5,C21),(A16,B6,C1),(A16,B6,C2),(A16,B6,C3),(A16, B6,C4),(A16,B6,C5),(A16,B6,C6),(A16,B6,C7),(A16,B6,C8),(A16, B6,C9),(A16,B6,C10),(A16,B6,C11),(A16,B6,C12),(A16,B6,C13), (A16,B6,C14),(A16,B6,C15),(A16,B6,C16),(A16,B6,C17),(A16,B 6,C18),(A16,B6,C19),(A16,B6,C20),(A16,B6,C21),(A17,B1,C1),
(A17,B1,C2),(A17,B1,C3),(A17,B1,C4),(A17,B1,C5),(A17,B1,C6), (A17,B1,C7),(A17,B1,C8),(A17,B1,C9),(A17,B1,C10),(A17,B1,C 11),(A17,B1,C12),(A17,B1,C13),(A17,B1,C14),(A17,B1,C15),(A1 7,B1,C16),(A17,B1,C17),(A17,B1,C18),(A17,B1,C19),(A17,B1,C20), (A17,B1, C21),(A17,B2,C1),(A17,B2,C2),(A17,B2,C3),(A17,B2, C4),(A17,B2,C5),(A17,B2,C6),(A17,B2,C7),(A17,B2,C8),(A17,B 2,C9),(A17,B2,C10),(A17,B2,C11),(A17,B2,C12),(A17,B2,C13),(A 17,B2,C14),(A17,B2,C15),(A17,B2,C16),(A17,B2,C17),(A17,B2, C18),(A17,B2,C19),(A17,B2,C20),(A17,B2,C21),(A17,B3,C1),(A1 7,B3,C2),(A17,B3,C3),(A17,B3,C4),(A17,B3,C5),(A17,B3,C6),(A 17,B3,C7),(A17,B3,C8),(A17,B3,C9),(A17,B3,C10),(A17,B3,C11), (A17,B3,C12),(A17,B3,C13),(A17,B3,C14),(A17,B3,C15),(A17,B 3,C16),(A17,B3,C17),(A17,B3,C18),(A17,B3,C19),(A17,B3,C20), (A17,B3,C21),(A17,B4,C1),(A17,B4,C2),(A17,B4,C3),(A17,B4,C4), (A17,B4,C5),(A17,B4,C6),(A17,B4,C7),(A17,B4,C8),(A17,B4,C 9),(A17,B4,C10),(A17,B4,C11),(A17,B4,C12),(A17,B4,C13),(A17, B4,C14),(A17,B4,C15),(A17,B4,C16),(A17,B4,C17),(A17,B4,C18), (A17,B4,C19),(A17,B4,C20),(A17,B4,C21),(A17,B5,C1),(A17,B 5,C2),(A17,B5,C3),(A17,B5,C4),(A17,B5,C5),(A17,B5,C6),(A17, B5,C7),(A17,B5,C8),(A17,B5,C9),(A17,B5,C10),(A17,B5,C11),(A 17,B5,C12),(A17,B5,C13),(A17,B5,C14),(A17,B5,C15),(A17,B5,C 16),(A17,B5,C17),(A17,B5,C18),(A17,B5,C19),(A17,B5,C20),(A1 7,B5,C21),(A17,B6,C1),(A17,B6,C2),(A17,B6,C3),(A17,B6,C4),(A 17,B6,C5),(A17,B6,C6),(A17,B6,C7),(A17,B6,C8),(A17,B6,C9), (A17,B6,C10),(A17,B6,C11),(A17,B6,C12),(A17,B6,C13),(A17,B6, C14),(A17,B6,C15),(A17,B6,C16),(A17,B6,C17),
(A17,B6,C18),(A17,B6,C19),(A17,B6,C20),(A17,B6,C21),(A18,B1, C1),(A18,B1,C2),(A18,B1,C3),(A18,B1,C4),(A18,B1,C5),(A18,B 1,C6),(A18,B1,C7),(A18,B1,C8),(A18,B1,C9),(A18,B1,C10),(A18, B1,C11),(A18,B1,C12),(A18,B1,C13),(A18,B1,C14),(A18,B1,C15), (A18,B1,C16),(A18,B1,C17),(A18,B1,C18),(A18,B1,C19),(A18, B1,C20),(A18,B1, C21),(A18,B2,C1),(A18,B2,C2),(A18,B2,C3),(A 18,B2,C4),(A18,B2,C5),(A18,B2,C6),(A18,B2,C7),(A18,B2,C8),(A 18,B2,C9),(A18,B2,C10),(A18,B2,C11),(A18,B2,C12),(A18,B2,C 13),(A18,B2,C14),(A18,B2,C15),(A18,B2,C16),(A18,B2,C17),(A8,B2,C18),(A18,B2,C19),(A18,B2,C20),(A18,B2,C21),(A18,B3,C1), (A18,B3,C2),(A18,B3,C3),(A18,B3,C4),(A18,B3,C5),(A18,B3,C 6),(A18,B3,C7),(A18,B3,C8),(A18,B3,C9),(A18,B3,C10),(A18,B3, C11),(A18,B3,C12),(A18,B3,C13),(A18,B3,C14),(A18,B3,C15), A18,B3,C16),(A18,B3,C17),(A18,B3,C18),(A18,B3,C19),(A18,B3, C20),(A18,B3,C21),(A18,B4,C1),(A18,B4,C2),(A18,B4,C3),(A18, B4,C4),(A18,B4,C5),(A18,B4,C6),(A18,B4,C7),(A18,B4,C8),(A18, B4,C9),(A18,B4,C10),(A18,B4,C11),(A18,B4,C12),(A18,B4,C13), (A18,B4,C14),(A18,B4,C15),(A18,B4,C16),(A18,B4,C17),(A18,B 4,C18),(A18,B4,C19),(A18,B4,C20),(A18,B4,C21),(A18,B5,C1),
(A18,B5,C2),(A18,B5,C3),(A18,B5,C4),(A18,B5,C5),(A18,B5,C6), (A18,B5,C7),(A18,B5,C8),(A18,B5,C9),(A18,B5,C10),(A18,B5,C 11),(A18,B5,C12),(A18,B5,C13),(A18,B5,C14),(A18,B5,C15),(A1 8,B5,C16),(A18,B5,C17),(A18,B5,C18),(A18,B5,C19),(A18,B5,C20), (A18,B5,C21),(A18,B6,C1),(A18,B6,C2),(A18,B6,C3),(A18,B6, C4),(A18,B6,C5),(A18,B6,C6),(A18,B6,C7),(A18,B6,C8),(A18,B 6,C9),(A18,B6,C10),(A18,B6,C11),(A18,B6,C12),(A18,B6,C13),(A 18,B6,C14),(A18,B6,C15),(A18,B6,C16),(A18,B6,C17),(A18,B6, C18),(A18,B6,C19),(A18,B6,C20),(A18,B6,C21),(A19,B1,C1),(A19,B1,C2),(A19,B1,C3),(A19,B1,C4),(A19,B1,C5),(A19,B1,C6),(A 19,B1,C7),(A19,B1,C8),(A19,B1,C9),(A19,B1,C10),(A19,B1,C11), (A19,B1,C12),(A19,B1,C13),(A19,B1,C14),(A19,B1,C15),(A19,B 1,C16),(A19,B1,C17),(A19,B1,C18),(A19,B1,C19),(A19,B1,C20), (A19,B1, C21),(A19,B2,C1),(A19,B2,C2),(A19,B2,C3),(A19,B2,C4), (A19,B2,C5),(A19,B2,C6),(A19,B2,C7),(A19,B2,C8),(A19,B2,C 9),(A19,B2,C10),(A19,B2,C11),(A19,B2,C12),(A19,B2,C13),(A19, B2,C14),(A19,B2,C15),(A19,B2,C16),(A19,B2,C17),(A19,B2,C18), (A19,B2,C19),(A19,B2,C20),(A19,B2,C21),(A19,B3,C1),(A19,B 3,C2),(A19,B3,C3),(A19,B3,C4),(A19,B3,C5),(A19,B3,C6),(A19, B3,C7),(A19,B3,C8),(A19,B3,C9),(A19,B3,C10),(A19,B3,C11),(A 19,B3,C12),(A19,B3,C13),(A19,B3,C14),(A19,B3,C15),(A19,B3,C 16),(A19,B3,C17),(A19,B3,C18),(A19,B3,C19),(A19,B3,C20),(A1 9,B3,C21),(A19,B4,C1),(A19,B4,C2),(A19,B4,C3),(A19,B4,C4),(A 19,B4,C5),(A19,B4,C6),(A19,B4,C7),(A19,B4,C8),(A19,B4,C9), (A19,B4,C10),(A19,B4,C11),(A19,B4,C12),(A19,B4,C13),(A19,B4, C14),(A19,B4,C15),(A19,B4,C16),(A19,B4,C17),
(A19,B4,C18),(A19,B4,C19),(A19,B4,C20),(A19,B4,C21),(A19,B5, C1),(A19,B5,C2),(A19,B5,C3),(A19,B5,C4),(A19,B5,C5),(A19,B 5,C6),(A19,B5,C7),(A19,B5,C8),(A19,B5,C9),(A19,B5,C10),(A19, B5,C11),(A19,B5,C12),(A19,B5,C13),(A19,B5,C14),(A19,B5,C15), (A19,B5,C16),(A19,B5,C17),(A19,B5,C18),(A19,B5,C19),(A19, B5,C20),(A19,B5,C21),(A19,B6,C1),(A19,B6,C2),(A19,B6,C3),(A 19,B6,C4),(A19,B6,C5),(A19,B6,C6),(A19,B6,C7),(A19,B6,C8),(A19,B6,C9),(A19,B6,C10),(A19,B6,C11),(A19,B6,C12),(A19,B6,C 13),(A19,B6,C14),(A19,B6,C15),(A19,B6,C16),(A19,B6,C17),(A1 9,B6,C18),(A19,B6,C19),(A19,B6,C20),(A19,B6,C21),(A20,B1,C1), (A20,B1,C2),(A20,B1,C3),(A20,B1,C4),(A20,B1,C5),(A20,B1,C 6),(A20,B1,C7),(A20,B1,C8),(A20,B1,C9),(A20,B1,C10),(A20,B1, C11),(A20,B1,C12),(A20,B1,C13),(A20,B1,C14),(A20,B1,C15),(A 20,B1,C16),(A20,B1,C17),(A20,B1,C18),(A20,B1,C19),(A20,B1, C20),(A20,B1, C21),(A20,B2,C1),(A20,B2,C2),(A20,B2,C3),(A20, B2,C4),(A20,B2,C5),(A20,B2,C6),(A20,B2,C7),(A20,B2,C8),(A20, B2,C9),(A20,B2,C10),(A20,B2,C11),(A20,B2,C12),(A20,B2,C13), (A20,B2,C14),(A20,B2,C15),(A20,B2,C16),(A20,B2,C17),(A20,B 2,C18),(A20,B2,C19),(A20,B2,C20),(A20,B2,C21),(A20,B3,C1),
(A20,B3,C2),(A20,B3,C3),(A20,B3,C4),(A20,B3,C5),(A20,B3,C6), (A20,B3,C7),(A20,B3,C8),(A20,B3,C9),(A20,B3,C10),(A20,B3,C 11),(A20,B3,C12),(A20,B3,C13),(A20,B3,C14),(A20,B3,C15),(A2 0,B3,C16),(A20,B3,C17),(A20,B3,C18),(A20,B3,C19),(A20,B3,C20), (A20,B3,C21),(A20,B4,C1),(A20,B4,C2),(A20,B4,C3),(A20,B4, C4),(A20,B4,C5),(A20,B4,C6),(A20,B4,C7),(A20,B4,C8),(A20,B 4,C9),(A20,B4,C10),(A20,B4,C11),(A20,B4,C12),(A20,B4,C13),(A 20,B4,C14),(A20,B4,C15),(A20,B4,C16),(A20,B4,C17),(A20,B4, C18),(A20,B4,C19),(A20,B4,C20),(A20,B4,C21),(A20,B5,C1),(A2 0,B5,C2),(A20,B5,C3),(A20,B5,C4),(A20,B5,C5),(A20,B5,C6),(A 20,B5,C7),(A20,B5,C8),(A20,B5,C9),(A20,B5,C10),(A20,B5,C11), (A20,B5,C12),(A20,B5,C13),(A20,B5,C14),(A20,B5,C15),(A20,B 5,C16),(A20,B5,C17),(A20,B5,C18),(A20,B5,C19),(A20,B5,C20), (A20,B5,C21),(A20,B6,C1),(A20,B6,C2),(A20,B6,C3),(A20,B6,C4), (A20,B6,C5),(A20,B6,C6),(A20,B6,C7),(A20,B6,C8),(A20,B6,C 9),(A20,B6,C10),(A20,B6,C11),(A20,B6,C12),(A20,B6,C13),(A20, B6,C14),(A20,B6,C15),(A20,B6,C16),(A20,B6,C17),(A20,B6,C18), (A20,B6,C19),(A20,B6,C20),(A20,B6,C21),(A21,B1,C1),(A21,B 1,C2),(A21,B1,C3),(A21,B1,C4),(A21,B1,C5),(A21,B1,C6),(A21, B1,C7),(A21,B1,C8),(A21,B1,C9),(A21,B1,C10),(A21,B1,C11),(A 21,B1,C12),(A21,B1,C13),(A21,B1,C14),(A21,B1,C15),(A21,B1,C 16),(A21,B1,C17),(A21,B1,C18),(A21,B1,C19),(A21,B1,C20),(A2 1,B1, C21),(A21,B2,C1),(A21,B2,C2),(A21,B2,C3),(A21,B2,C4),(A 21,B2,C5),(A21,B2,C6),(A21,B2,C7),(A21,B2,C8),(A21,B2,C9), (A21,B2,C10),(A21,B2,C11),(A21,B2,C12),(A21,B2,C13),(A21,B2, C14),(A21,B2,C15),(A21,B2,C16),(A21,B2,C17),
(A21,B2,C18),(A21,B2,C19),(A21,B2,C20),(A21,B2,C21),(A21,B3, C1),(A21,B3,C2),(A21,B3,C3),(A21,B3,C4),(A21,B3,C5),(A21,B 3,C6),(A21,B3,C7),(A21,B3,C8),(A21,B3,C9),(A21,B3,C10),(A21, B3,C11),(A21,B3,C12),(A21,B3,C13),(A21,B3,C14),(A21,B3,C15), (A21,B3,C16),(A21,B3,C17),(A21,B3,C18),(A21,B3,C19),(A21, B3,C20),(A21,B3,C21),(A21,B4,C1),(A21,B4,C2),(A21,B4,C3),(A 21,B4,C4),(A21,B4,C5),(A21,B4,C6),(A21,B4,C7),(A21,B4,C8),(A 21,B4,C9),(A21,B4,C10),(A21,B4,C11),(A21,B4,C12),(A21,B4,C 13),(A21,B4,C14),(A21,B4,C15),(A21,B4,C16),(A21,B4,C17),(A2 1,B4,C18),(A21,B4,C19),(A21,B4,C20),(A21,B4,C21),(A21,B5,C1), (A21,B5,C2),(A21,B5,C3),(A21,B5,C4),(A21,B5,C5),(A21,B5,C 6),(A21,B5,C7),(A21,B5,C8),(A21,B5,C9),(A21,B5,C10),(A21,B5, C11),(A21,B5,C12),(A21,B5,C13),(A21,B5,C14),(A21,B5,C15),(A 21,B5,C16),(A21,B5,C17),(A21,B5,C18),(A21,B5,C19),(A21,B5, C20),(A21,B5,C21),(A21,B6,C1),(A21,B6,C2),(A21,B6,C3),(A21, B6,C4),(A21,B6,C5),(A21,B6,C6),(A21,B6,C7),(A21,B6,C8),(A21, B6,C9),(A21,B6,C10),(A21,B6,C11),(A21,B6,C12),(A21,B6,C13), (A21,B6,C14),(A21,B6,C15),(A21,B6,C16),(A21,B6,C17),(A21,B 6,C18),(A21,B6,C19),(A21,B6,C20),(A21,B6,C21),(A22,B1,C1),
(A22,B1,C2),(A22,B1,C3),(A22,B1,C4),(A22,B1,C5),(A22,B1,C6), (A22,B1,C7),(A22,B1,C8),(A22,B1,C9),(A22,B1,C10),(A22,B1,C 11),(A22,B1,C12),(A22,B1,C13),(A22,B1,C14),(A22,B1,C15),(A2 2,B1,C16),(A22,B1,C17),(A22,B1,C18),(A22,B1,C19),(A22,B1,C20), (A22,B1, C21),(A22,B2,C1),(A22,B2,C2),(A22,B2,C3),(A22,B2, C4),(A22,B2,C5),(A22,B2,C6),(A22,B2,C7),(A22,B2,C8),(A22,B 2,C9),(A22,B2,C10),(A22,B2,C11),(A22,B2,C12),(A22,B2,C13),(A 22,B2,C14),(A22,B2,C15),(A22,B2,C16),(A22,B2,C17),(A22,B2, C18),(A22,B2,C19),(A22,B2,C20),(A22,B2,C21),(A22,B3,C1),(A2 2,B3,C2),(A22,B3,C3),(A22,B3,C4),(A22,B3,C5),(A22,B3,C6),(A 22,B3,C7),(A22,B3,C8),(A22,B3,C9),(A22,B3,C10),(A22,B3,C11), (A22,B3,C12),(A22,B3,C13),(A22,B3,C14),(A22,B3,C15),(A22,B 3,C16),(A22,B3,C17),(A22,B3,C18),(A22,B3,C19),(A22,B3,C20), (A22,B3,C21),(A22,B4,C1),(A22,B4,C2),(A22,B4,C3),(A22,B4,C4), (A22,B4,C5),(A22,B4,C6),(A22,B4,C7),(A22,B4,C8),(A22,B4,C 9),(A22,B4,C10),(A22,B4,C11),(A22,B4,C12),(A22,B4,C13),(A22, B4,C14),(A22,B4,C15),(A22,B4,C16),(A22,B4,C17),(A22,B4,C18), (A22,B4,C19),(A22,B4,C20),(A22,B4,C21),(A22,B5,C1),(A22,B 5,C2),(A22,B5,C3),(A22,B5,C4),(A22,B5,C5),(A22,B5,C6),(A22, B5,C7),(A22,B5,C8),(A22,B5,C9),(A22,B5,C10),(A22,B5,C11),(A 22,B5,C12),(A22,B5,C13),(A22,B5,C14),(A22,B5,C15),(A22,B5,C 16),(A22,B5,C17),(A22,B5,C18),(A22,B5,C19),(A22,B5,C20),(A2 2,B5,C21),(A22,B6,C1),(A22,B6,C2),(A22,B6,C3),(A22,B6,C4),(A 22,B6,C5),(A22,B6,C6),(A22,B6,C7),(A22,B6,C8),(A22,B6,C9), (A22,B6,C10),(A22,B6,C11),(A22,B6,C12),(A22,B6,C13),(A22,B6, C14),(A22,B6,C15),(A22,B6,C16),(A22,B6,C17),
(A22,B6,C18),(A22,B6,C19),(A22,B6,C20),(A22,B6,C21),(A23,B1, C1),(A23,B1,C2),(A23,B1,C3),(A23,B1,C4),(A23,B1,C5),(A23,B 1,C6),(A23,B1,C7),(A23,B1,C8),(A23,B1,C9),(A23,B1,C10),(A23, B1,C11),(A23,B1,C12),(A23,B1,C13),(A23,B1,C14),(A23,B1,C15), (A23,B1,C16),(A23,B1,C17),(A23,B1,C18),(A23,B1,C19),(A23, B1,C20),(A23,B1, C21),(A23,B2,C1),(A23,B2,C2),(A23,B2,C3),(A 23,B2,C4),(A23,B2,C5),(A23,B2,C6),(A23,B2,C7),(A23,B2,C8), A23,B2,C9),(A23,B2,C5), (A23,B2,C6), (A23,B2,C7),(A23,B2,C 13),(A23,B2,C14),(A23,B2,C15),(A23,B2,C16),(A23,B2,C17),(A2 3,B2, C18),(A23,B2, C19),(A23,B2,C20),(A23,B2,C21),(A23,B3,C1), A23,B3,C2),(A23,B3,C3),(A23,B3,C4),(A23,B3,C5),(A23,B3,C 6),(A23,B3,C7),(A23,B3,C8),(A23,B3,C9),(A23,B3,C10),(A23,B3, C11),(A23,B3,C12),(A23,B3,C13),(A23,B3,C14),(A23,B3,C15), (A 23,B3,C16),(A23,B3,C17),(A23,B3,C18),(A23,B3,C19),(A23,B3, C20),(A23,B3,C 21),(A23,B4,C1), (A23,B 4,C2),(A23,B 4,C3),(A23, B4,C4),(A23,B4,C5),(A23,B4,C6),(A23,B4,C7),(A23,B4,C8),(A23, B4,C9),(A23,B4,C10),(A23,B4,C11), (A23,B4,C12),(A23,B4,C13), (A23,B4,C14),(A23,B4,C15),(A23,B4,C16),(A23,B4,C17),(A23,B 4,C18),(A23,B4,C19),(A23,B4,C20),(A23,B4,C21),(A23,B5,C1), (A23,B5,C2),(A23,B5,C3),(A23,B5,C4),(A23,B5,C5),(A23,B5,C6), (A23,B5,C7),(A23,B5,C8),(A23,B5,C9),(A23,B5,C10),(A23,B5, C 11),(A23,B5,C12),(A23,B5,C13),(A23,B5,C14),(A23,B5,C15),(A2 3,B5,C16),(A23,B5,C17),(A23,B5,C18),(A23,B5,C19),(A23,B5,C20), (A23,B5,C21),(A23,B6,C1),(A23,B6,C2),(A23,B6,C3), (A23,B6, C4),(A23,B6,C5),(A23,B6,C6),(A23,B6,C7),(A23,B6,C8),(A23,B 6,C9),(A23,B6,C10),(A23,B6,C11),(A23,B6,C12),(A23,B6,C13),(A 23,B6,C14),(A23,B6,C15),(A23,B6,C16),(A23,B6,C17),(A23,B6, C18),(A23,B6,C19),(A23,B6,C20),(A23,B6,C21),(A24,B1,C1),(A2 4,B1,C2),(A24,B1,C3),(A24,B1,C4),(A24,B1,C5),(A24,B1,C6), (A 24,B1,C7),(A24,B1,C8), (A24,B1,C9),(A24,B1,C10),(A24,B1,C11), (A24,B1,C12),(A24,B1,C13),(A24,B1,C14),(A24,B1,C15),(A24,B 1,C16),(A24,B1,C17),(A24,B1,C18),(A24,B1,C19),(A24,B1,C20), (A24,B1,C21),(A24,B2,C1), (A24,B2,C2),(A24,B2,C3),(A24,B2,C4), (A24,B2,C5),(A24,B2,C6), (A24,B2,C7),(A24,B2,C8),(A24,B2,C 9), (A24,B2,C10),(A24,B2,C11),(A24,B2,C12),(A24,B2,C13),(A24, B2,C14),(A24,B2,C15),(A24,B2,C16), (A24,B2,C17), (A24,B2,C18), (A24,B2,C19), (A24,B2,C20),(A24,B2,C21),(A24,B3,C1), (A24,B 3,C2),(A24,B3,C3),(A24,B3,C4),(A24,B3,C5),(A24,B3,C6),(A24, B3,C7),(A24,B3,C8),(A24,B3,C9),(A24,B3,C10), (A24,B3,C11), (A 24,B3,C12),(A24,B3,C13),(A24,B3,C14),(A24,B3,C15),(A24,B3,C

16),(A24,B3,C17),(A24,B3,C18), (A24,B3,C19),(A24,B3,C20),(A2 4,B3,C21),(A24,B4,C1), (A24,B4,C2),(A24,B4,C3),(A24,B4,C4),

(A24,B4,C5),(A24,B4,C6),(A24,B4,C7),(A24,B4,C8),(A24,B4,C9), (A24,B4,C10),(A24,B4,C11),(A24,B4,C12),(A24,B4,C13),(A24,B 4,C14),(A24,B4,C15),(A24,B4,C16),(A24,B4,C17),(A24,B4,C118, (A24,B4,C19),(A24,B4,C20),(A24,B4,C21),(A24,B5,C1),(A24,B5, C2),(A24,B5,C3),(A24,B5,C4),(A24,B5,C5, (A24,B5,C6),(A24,B5, C7),(A24,B5,C8),(A24,B5,C9),(A24,B5,C10),(A24,B5,C11),(A24, B5,C12),(A24,B5,C13),(A24,B5,C14),(A24,B5,C15),(A24,B5,C16), (A24,B5,C17),(A24,B5,C18),(A24,B5,C19),(A24,B5,C20),(A24, B5,C21),(A24,B6,C1),(A24,B6,C2),(A24,B6,C3),(A24,B6,C4),(A2 4,B6,C5),(A24,B6,C6),(A24,B6,C7),(A24,B6,C8),(A24,B6,C9),(A 24,B6,C10),(A24,B6,C11),(A24,B6,C12),(A24,B6,C13),(A24,B6,C 14),(A24,B6,C15),(A24,B6,C16),(A24,B6,C17),(A24,B6,C18),(A2 4,B6,C19),(A24,B6,C20),(A24,B6,C21),(A25,B1,C1),(A25,B1,C2), (A25,B1,C3),(A25,B1,C4),(A25,B1,C5),(A25,B1,C6),(A25,B1,C7), (A25,B1,C8),(A25,B1,C9),(A25,B1,C10),(A25,B1, C11),(A25,B1, C12),(A25,B1,C13),(A25,B1,C14),(A25,B1,C15),(A25,B1,C16),(A 25,B1,C17),(A25,B1,C18),(A25,B1,C19),(A25,B1,C20),(A25,B1, C21),(A25,B2,C1),(A25,B2,C2),(A25,B2,C3),(A25,B2,C4),(A25,B 2,C5),(A25,B2,C6),(A25,B2,C7),(A25,B2,C8),(A25,B2,C9),(A25, B2,C10),(A25,B2,C11),(A25,B2,C12),(A25,B2,C13),(A25,B2,C14), (A25,B2,C15),(A25,B2,C16),(A25,B2,C17),(A25,B2,C18),(A25,B 2,C19),(A25,B2,C20),(A25,B2,C21),(A25,B3,C1),(A25,B3,C2),(A 25,B3,C3),(A25,B3,C4),(A25,B3,C5),(A25,B3,C6),(A25,B3,C7),
(A25,B3,C8),(A25,B3,C9),(A25,B3,C10),(A25,B3,C11),(A25,B3,C 12),(A25,B3,C13),(A25,B3,C14),(A25,B3,C15),(A25,B3,C16),(A2 5,B3,C17),(A25,B3,C18),(A25,B3,C19),(A25,B3,C20),(A25,B3,C21), (A25,B4,C1),(A25,B4,C2),(A25,B4,C3),(A25,B4,C4),(A25,B4, C5),(A25,B4,C6),(A25,B4,C7),(A25,B4,C8),(A25,B4,C9),(A25,B4, C10),(A25,B4,C11),(A25,B4,C12),(A25,B4,C13),(A25,B4,C14),(A 25,B4,C15),(A25,B4,C16),(A25,B4,C17),(A25,B4,C18),(A25,B4, C19),(A25,B4,C20),(A25,B4,C21),(A25,B5,C1),(A25,B5,C2),(A25, B5,C3),(A25,B5,C4),(A25,B5,C5),(A25,B5,C6),(A25,B5,C7),(A2 5,B5,C8),(A25,B5,C9),(A25,B5,C10),(A25,B5,C11),(A25,B5,C12), (A25,B5,C13),(A25,B5,C14),(A25,B5,C15),(A25,B5,C16),(A25,B 5,C17),(A25,B5,C18),(A25,B5,C19),(A25,B5,C20),(A25,B5,C21), (A25,B6,C1),(A25,B6,C2),(A25,B6,C3),(A25,B6,C4),(A25,B6,C5), (A25,B6,C6),(A25,B6,C7),(A25,B6,C8),(A25,B6,C9),(A25,B6,C10), (A25,B6,C11),(A25,B6,C12),(A25,B6,C13),(A25,B6,C14),(A25, B6,C15),(A25,B6,C16),(A25,B6,C17),(A25,B6,C18),(A25,B6,C19), (A25,B6,C20),(A25,B6,C21),(A26,B1,C1),(A26,B1,C2),(A26,B1, C3),(A26,B1,C4),(A26,B1,C5),(A26,B1,C6),(A26,B1,C7),(A26,B 1,C8),(A26,B1,C9),(A26,B1,C10),(A26,B1,C11),(A26,B1,C12),
(A26,B1,C13),(A26,B1,C14),(A26,B1,C15),(A26,B1,C16),(A26,B1, C17),(A26,B1,C18),(A26,B1,C19),(A26,B1,C20),(A26,B1,C21),(A 26,B2,C1),(A26,B2,C2),(A26,B2,C3),(A26,B2,C4),(A26,B2,C5), (A26,B2,C6),(A26,B2,C7),(A26,B2,C8),(A26,B2,C9),(A26,B2,C10), (A26,B2,C11),(A26,B2,C12),(A26,B2,C13),(A26,B2,C14),(A26, B2,C15),(A26,B2,C16),(A26,B2,C17),(A26,B2,C18),(A26,B2,C19), (A26,B2,C20),(A26,B2,C21),(A26,B3,C1),(A26,B3,C2),(A26,B3, C3),(A26,B3,C4),(A26,B3,C5),(A26,B3,C6),(A26,B3,C7),(A26,B3, C8),(A26,B3,C9),(A26,B3,C10),(A26,B3,C11),(A26,B3,C12),(A2 6,B3,C13),(A26,B3,C14),(A26,B3,C15),(A26,B3,C16),(A26,B3,C17), (A26,B3,C18),(A26,B3,C19),(A26,B3,C20),(A26,B3,C21),(A26, B4,C1),(A26,B4,C2),(A26,B4,C3),(A26,B4,C4),(A26,B4,C5),(A2 6,B4,C6),(A26,B4,C7),(A26,B4,C8),(A26,B4,C9),(A26,B4,C10),(A 26,B4,C11),(A26,B4,C12),(A26,B4,C13),(A26,B4,C14),(A26,B4, C15),(A26,B4,C16),(A26,B4,C17),(A26,B4,C18),(A26,B4,C19),(A 26,B4,C20),(A26,B4,C21),(A26,B5,C1),(A26,B5,C2),(A26,B5,C3), (A26,B5,C4),(A26,B5,C5),(A26,B5,C6),(A26,B5,C7),(A26,B5,C8), (A26,B5,C9),(A26,B5,C10),(A26,B5,C11),(A26,B5,C12),(A26,B 5,C13),(A26,B5,C14),(A26,B5,C15),(A26,B5,C16),(A26,B5,C17), (A26,B5,C18),(A26,B5,C19),(A26,B5,C20),(A26,B5,C21),(A26,B6, C1),(A26,B6,C2), (A26,B6,C3),(A26,B6,C4),(A26,B6,C5),(A26,B6,C6),(A26,B6,C7), (A26,B6,C8),(A26,B6,C9),(A26,B6,C10),(A26,B6,C11),(A26,B6, C12),(A26,B6,C13),(A26,B6,C14),(A26,B6,C15),(A26,B6,C16),(A 26,B6,C17),(A26,B6,C18),(A26,B6,C19),(A26,B6,C20),(A26,B6,C 21),(A27,B1,C1),(A27,B1,C2),(A27,B1,C3),(A27,B1,C4),(A27,B1, C5),(A27,B1,C6),(A27,B1,C7),(A27,B1,C8),(A27,B1,C9),(A27,B 1,C10),(A27,B1,C11),(A27,B1,C12),(A27,B1,C13),(A27,B1,C14), (A27,B1,C15),(A27,B1,C16),(A27,B1,C17),(A27,B1,C18),(A27,B1, C19),(A27,B1,C20),(A27,B1, C21),(A27,B2,C1),(A27,B2,C2),(A2 7,B2,C3),(A27,B2,C4),(A27,B2,C5),(A27,B2,C6),(A27,B2,C7),(A27,B2,C8),(A27,B2,C9),(A27,B2,C10),(A27,B2,C11),(A27,B2,C12), (A27,B2,C13),(A27,B2,C14),(A27,B2,C15),(A27,B2,C16),(A27, B2,C17)/(A27,B2,C18),(A27,B2,C19),(A27,B2,C20),(A27,B2,C21), (A27,B3,C1),(A27,B3,C2),(A27,B3,C3),(A27,B3,C4),(A27,B3,C5), (A27,B3,C6),(A27,B3,C7),(A27,B3,C8),(A27,B3,C9),(A27,B3,C 10),(A27,B3,C11),(A27,B3,C12),(A27,B3,C13),(A27,B3,C14),(A2 7,B3,C15),(A27,B3,C16),(A27,B3,C17),(A27,B3,C18),(A27,B3,C19), (A27,B3,C20),(A27,B3,C21),(A27,B4,C1),(A27,B4,C2),(A27,B 4,C3),(A27,B4,C4),(A27,B4,C5),(A27,B4,C6),(A27,B4,C7),(A27, B4,C8),(A27,B4,C9),(A27,B4,C10),(A27,B4,C11),(A27,B4,C12),(A 27,B4,C13),(A27,B4,C14),(A27,B4,C15),(A27,B4,C16),(A27,B4, C17),(A27,B4,C18),(A27,B4,C19),(A27,B4,C20),(A27,B4,C21),(A 27,B5,C1),(A27,B5,C2),(A27,B5,C3),(A27,B5,C4),(A27,B5,C5),
(A27,B5,C6),(A27,B5,C7),(A27,B5,C8),(A27,B5,C9),(A27,B5,C10), (A27,B5,C11),(A27,B5,C12),(A27,B5,C13),(A27,B5,C14),(A27, B5,C15),(A27,B5,C16),(A27,B5,C17),(A27,B5,C18),(A27,B5,C19), (A27,B5,C20),(A27,B5,C21),(A27,B6,C1),(A27,B6,C2),(A27,B6, C3),(A27,B6,C4),(A27,B6,C5),(A27,B6,C6),(A27,B6,C7),(A27,B6, C8),(A27,B6,C9),(A27,B6,C10),(A27,B6,C11),(A27,B6,C12),(A2 7,B6,C13),(A27,B6,C14),(A27,B6,C15),(A27,B6,C16),(A27,B6,C17), (A27,B6,C18),(A27,B6,C19),(A27,B6,C20),(A27,B6,C21).

The present invention is illustrated in more detail by the following Examples but should not be construed to be limited thereto. Besides, each abbreviation means the followings;

Me: methyl

Et: ethyl

Bu: butyl

Ac: acetyl

Ph: phenyl

DMF: dimethylformamide

TsOH: p-toluenesulfonic acid

rt: room temperature

### EXAMPLES

### Example 1

### Preparation of N-(4-tert-butylphenyl)-4-(2-methyl-1,3-dioxane-2-yl)benzamide(Ia-2)

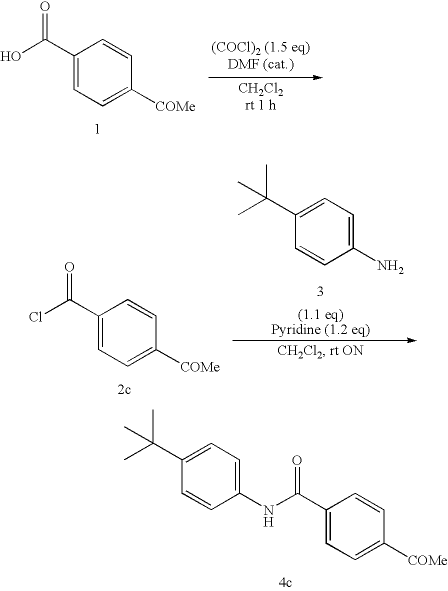

(the 1^{st} step)

To a mixture of 4-acetylbenzoic acid (compound 1: 1.64 g, 10 mmole), oxalyl chloride (1.07 mL, 12 mmole) and dichloromethane (30 mL) was added a drop of N,N-dimethylformamide, and the mixture was stirred at rt for 1 hour and then heated to reflux for 1 hour. The reaction mixture was concentrated in vacuo to give a crude product of 4-acetylbenzoyl chloride (compound 2c).

To a mixture of 4-tert-butyl aniline (compound 3: 1.64 g, 11 mmole), pyridine (1.03 g, 13 mmole) and dichloromethane (20 mL), was added dropwise the dichloromethane (10 mL) solution of 4-acetyl benzoyl chloride obtained above under ice-cooling over 10 minutes, and the resulting mixture was stirred at rt for 2 hours. Water (150 mL) and 1M HCl (20 mL) were added to the reaction mixture, and the product was extracted with ethyl acetate (200 mL). The extract was washed with saturated brine (150 mL), dried over MgSO₄ and concentrated in vacuo. The residue was recrystallized from ethyl acetate/n-hexane to give N-(4-tert-butylphenyl)-4-acetylbenzamide (compound 4c: 2.24 g, yield 76%) as a pale yellow crystalline. Mp. 183-184° C.

### (the 2^{nd} Step)

### Preparation of N-(4-tert-butylphenyl)-4-(2-methyl-1,3-dioxane-2-yl)benzamide (Ia-2)

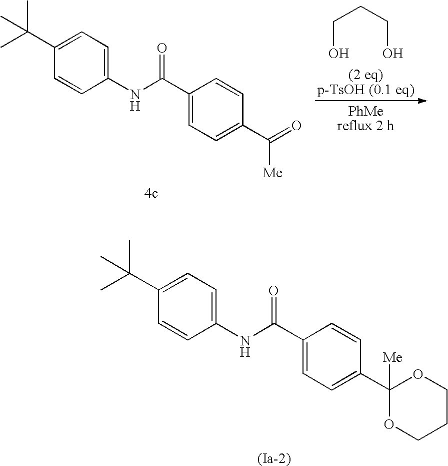

A mixture of N-(4-tert-butylphenyl)-4-acetylbenzamide (compound 4c: 0.15 g, 0.5 mmole), 1,3-propyleneglycol (0.08 g, 1 mmole), p-toluenesulfonic acid (0.01 g, 0.05 mmole) and toluene (2 mL) was refluxed under azeotropical distillation for 2 hours. To the reaction mixture, was added water (80 mL) and the product was extracted with ethyl acetate (100 mL). The extract was washed with saturated brine, dried over MgSO₄ and concentrated in vacuo. The resulting residue was purified by column chromatography on silica gel (ethyl acetate/n-hexane) and recrystallized from ethyl acetate/n-hexane to give N-(4-tert-butylphenyl)-4-(2-methyl-1,3-dioxane-2-yl)benzamide(compound 1a-2: 0.10 g, yield 57%) as a colorless crystalline. Mp. 148-149° C.

### Example 2

### Preparation of N-(4-tert-butylphenyl)-4-(1-methoxyiminoethyl)benzamide(Ij-4)

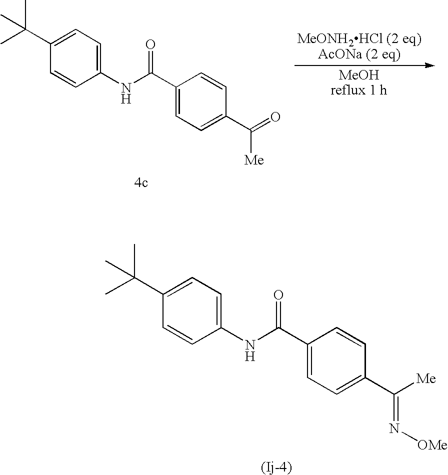

A mixture of N-(4-tert-butylphenyl)-4-acetylbenzamide(compound 4c: 0.15 g, 0.5 mmolre), methoxyamine hydrochloride (0.084 g, 11.0 mmole), sodium acetate (0.082 g, 11.0 mmole) and methanol (2 mL) was refluxed for 1 hour. To the reaction mixture was added water (80 mL), and the product was extracted with ethyl acetate (100 mL). The extract was washed with saturated brine, dried over MgSO₄ and concentrated in vacuo. The resulting residue was purified by column chromatography on silica gel(ethyl acetate/n-hexane) and recrystallized from ethyl acetate/n-hexane to give N-(4-tert-butylphenyl)-4-(1-methoxyiminoethyl)benzamide(Ij-4: 0.12 g, yield 74%) as a colorless crystalline. Mp. 154-155° C.

### Example 3

### Preparation of N-(4-tert-butylphenyl)-4-(2-thiazolinyl)benzamide(If-1)

### (the 1^{st} Step)

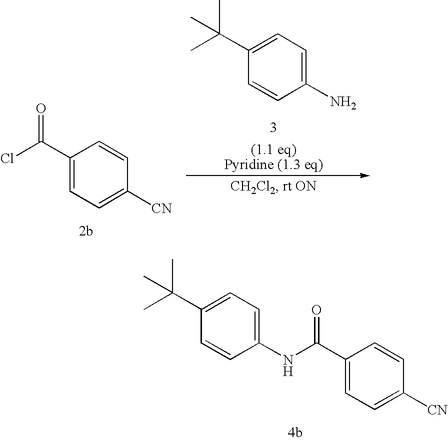

To a mixture of 4-tert-butyl aniline(compound 3: 1.64 g, 11 mmole), pyridine (1.03 g, 13 mmole) and dichloromethane (10 mL) was added a dichloromethane (10 mL) solution of 4-cyanobenzoyl chloride(compound 2b: 1.66 g, 10 mmole) under ice cooling over 5 minutes and stirred at rt for 2 hours. To the reaction mixture was added water (150 mL) and 1M HCl (20 mL), and the product was extracted with ethyl acetate (200 mL). The extract was washed with saturated brine, dried over MgSO₄ and concentrated in vacuo. The resulting residue was recrystallized from ethyl acetate/n-hexane to give N-(4-tert-butylphenyl)-4-cyanobenzamide(compound 4b: 2.52 g, yield 91%) as a colorless crystalline. Mp. 210-211° C.

### (the 2^{nd} Step)

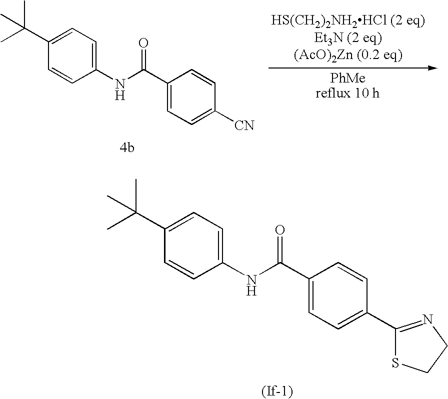

A mixture of N-(4-tert-butylphenyl)-4-cyanobenzamide (compound 4b: 0.14 g, 0.5 mmole), aminoethanethiol hydrochloride (0.11 g, 0.1 mmole), zinc acetate hydrate (0.02 g, 0.01 mmole) and toluene (2 mL) was refluxed under azeotropical distillation for 10 hours. To the reaction mixture was added water (80 mL), and the product was extracted with ethyl acetate (100 mL). The extract was washed with saturated brine, dried over MgSO₄ and concentrated in vacuo. The resulting residue was purified by column chromatography on silica gel(ethyl acetate/n-hexane) and recrystallized from ethyl acetate/n-hexane to give N-(4-tert-butylphenyl)-4-(2-thiazolinyl)benzamide(compound 1f-1: 0.13 g, yield 77%) as a light yellow crystalline. Mp. 176-177° C.

### Example 4

### Preparation of N-(4-trifluoromethylphenyl)-4-(4-ethoxy-3-isoxazolinyl)benzamide(Ig-1)

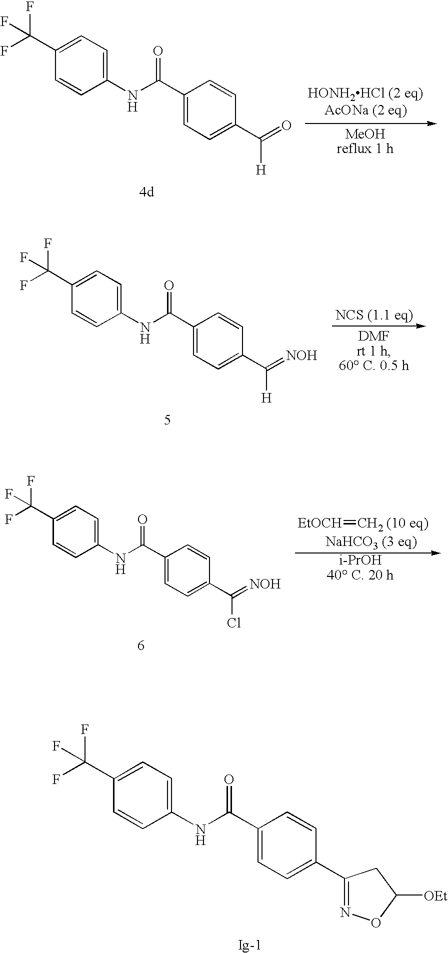

### (the 1^{st} Step)

A mixture of N-(4-trifluoromethylphenyl)-4-formylbenzamide(compound 4d; 0.88 g, 3.0 mmole), hydroxylamine hydrochloride (0.42 g, 6.0 mmole), sodium acetate (0.49 g6.0 mmole) and methanol (6 mL) was refluxed for 1 hour. To the reaction mixture was added water (100 mL), and the product was extracted with ethyl acetate (150 mL). The extract was washed with saturated brine (100 mL), dried over MgSO₄ and concentrated in vacuo. The resulting residue was recrystallized from ethyl acetate/n-hexane to give N-(4-trifluoromethylphenyl)-4-(hydroxyiminomethyl)benzamide(compound 5: 0.58 g, yield 63%) as a colorless crystalline. Mp. 221-222° C.

### (the 2^{nd} Step)

N-Chlorosuccinimide (0.088 g, 0.66 mmole) was added to a mixture of N-(4-trifluoromethylphenyl)-4-(hydroxyiminomethyl)benzamide(compound 5: 0.185 g, 0.6 mmole) and N,N-dimethylformamide (4 mL), and stirred at rt for 1 hour and at 60° C. for 0.5 hour. To the reaction mixture was added water (80 mL), and the product was extracted with ethyl acetate (100 mL). The extract was washed with saturated brine (100 mL), dried over MgSO₄ and concentrated in vacuo to give N-(4-trifluoromethylphenyl)-4-[chloro(hydroxyimino)methyl]benzamide(compound 6) as a crude product.

The crude product was dissolved in 2-propanol (3 mL) and stirred at 40° C. for 20 hours after ethylvinylether (0.43 g, 6.0 mmole) and sodium bicarbonate (0.15 g, 1.8 mmole) were added. To the reaction mixture was added water (80 mL), and the product was extracted with ethyl acetate (100 mL). The extract was washed with saturated brine (80 mL), dried over MgSO₄ and concentrated in vacuo. The resulting residue was recrystallized from ethyl acetate to give N-(4-trifluoromethylphenyl)-4-(4-ethoxy-3-isoxazolinyl)benzamide(compound 1 g-1: 0.12 g, yield 53%) as a colorless crystalline. Mp. 249-250° C.

### Example 5

### Preparation of N-(4-tert-butylphenyl)-4-(5-oxo-1,2,4-oxadiazolidine-3-yl)benzamide(Ih-1)

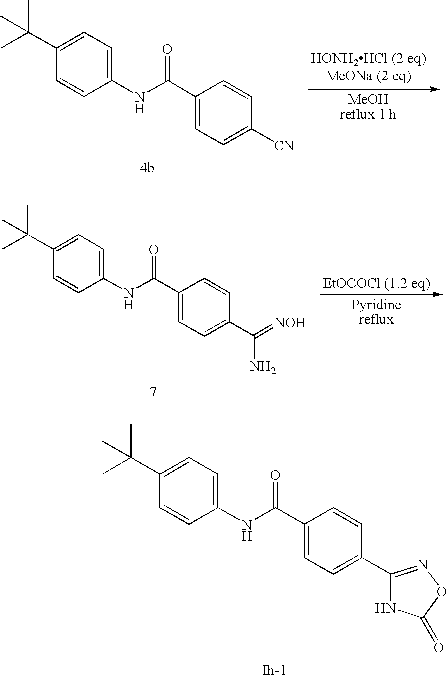

A mixture of hydroxylamine hydrochloride (0.14 g, 2.0 mmole) and methanol (4 mL) was neutralized by adding a 28% methanol solution of sodium methylate (0.39 g, 2.0 mmole) under ice cooling. To the resulting suspension was added N-(4-tert butylphenyl)-4-cyanobenzamide(compound 4b: 0.28 g, 1.0 mmole) and refluxed for 1 hour. To the reaction mixture was added water(80 mL), and the product was extracted with ethyl acetate (100 mL). The extract was washed with saturated brine (80 mL), dried over MgSO₄ and concentrated in vacuo to give N-(4-tert-butylphenyl)-4-[amino(hydroxyimino)methyl]benzamide(compound 7: 0.32 g, yield 100%) as a colorless crystalline.

The N-(4-tert-butylphenyl)-4-[amino(hydroxyimino)methyl]benzamide(compound 7: 0.16 g, 0.5 mmole) was dissolved in pyridine (2 mL), and ethyl chloroformate (0.06 mL, 0.6 mmole) was added to the solution under ice cooling. The resulting mixture was refluxed for 1 hour. To the reaction mixture was added water (80 mL) and 2M HCl (20 mL), and the product was extracted with ethyl acetate (100 mL). The extract was washed with saturated brine (80 mL), dried over MgSO4 and concentrated in vacuo. The resulting residue was recrystallized to give N-(4-tert-butylphenyl)-4-(5-oxo-1,2,4-oxadiazolidine-3-yl)benzamide(compound 1h-1: 0.13 g, yields 77%) as a colorless crystalline. Mp.>300° C.

### Example 6

### Preparation of N-(4-tert-butylphenyl)-4-(5,5-dimethyl-1,2,4-oxadiazolidine-3-yl)benzamide

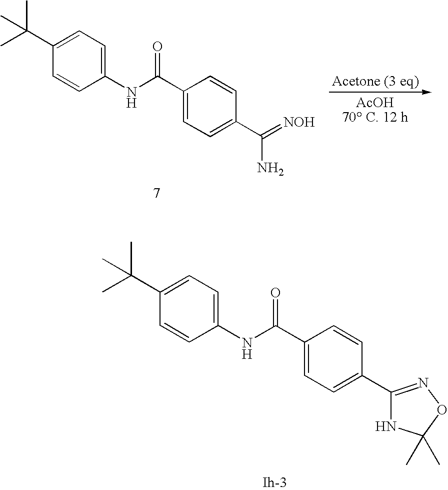

To a mixture of N-(4-tert-butylphenyl)-4-[amino(hydroxyimino)methyl]benzamide(compound 7: 0.16 g, 0.5 mmole) and acetic acid (2 mL) was added acetone (0.09 g, 1.5 mmole) and the mixture was stirred at 70° C. for 12 hours. To the reaction mixture were added water (40 mL) and a saturated aqueous solution of NaHCO₃(40 mL), and the product was extracted with ethyl acetate (100 mL). The extract was washed with saturated brine (80 mL), dried over MgSO₄ and concentrated in vacuo. The resulting residue was purified by column chromatography on silica gel(ethyl acetate/n-hexane) to give N-(4-tert-butylphenyl)-4-(5,5-dimethyl-1,2,4-oxazolidine-3-yl)benzamide(compound 1h-3: 0.05 g, yield 28%) as a colorless crystalline. Mp. 236-238° C.

### Example 7

Other compounds were synthesized in the same way as above. Their structure and physical constant were shown in the following tables.

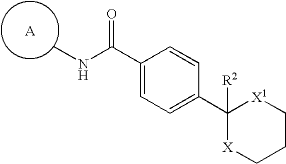

| Comp. | A | X | X¹ | R² | m.p. (° C.) |
|---|---|---|---|---|---|
| Ia-1 | 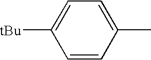 | O | O | H | 165-166 |
| Ia-2 | 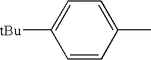 | O | O | Me | 148-149 |
| Ia-3 | 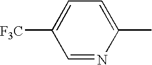 | O | O | H | 201-202 |

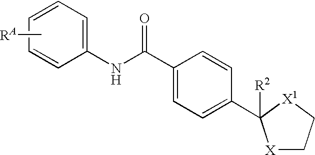

| Comp. | R^{A1} | X | X¹ | R² | m.p. (° C.) |
|---|---|---|---|---|---|
| Ib-1 | 4-tBu | S | NH | H | 119-120 |
| Ib-2 | 4-tBu | S | NH | Me | 152-153 |
| Ic-1 | 4-tBu | O | NH | Me | 138-139 |
| Id-1 | 4-CF₃ | NMe | NMe | H | 169-171 |
| Ie-1 | 4-tBu | O | O | H | 144-145 |
| Ie-2 | 4-tBu | O | O | Me | 180-189 |

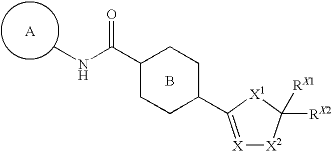

| Comp. | A | B | X | X¹ | X² | R^{X1} | R^{X2} | m.p. (° C.) |
|---|---|---|---|---|---|---|---|---|
| If-1 | 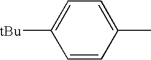 | 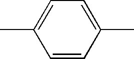 | N | S | CH₂ | H | H | 176-177 |
| If-2 | 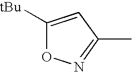 | 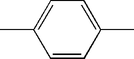 | N | S | CH₂ | H | H | 214-216 |
| If-3 | 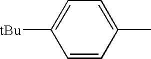 | 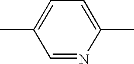 | N | S | CH₂ | H | H | 204-205 |
| If-4 | 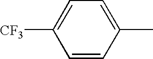 | 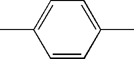 | N | S | CH₂ | H | H | 259-261 |
| Ig-1 | 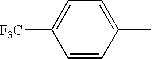 | 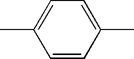 | N | CH₂ | O | H | OEt | 249-250 |
| Ih-1 | 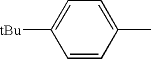 | 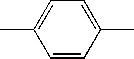 | N | NH | O | ═O | | >300 |
| Ih-2 | 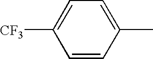 | 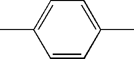 | N | NH | O | ═O | | >300 |
| Ih-3 | 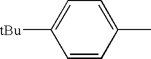 | 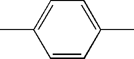 | N | NH | O | Me | Me | 236-238 |
| Ii-1 | 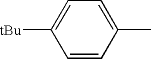 | 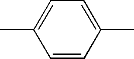 | N | O | CH₂ | H | H | 192-193 |

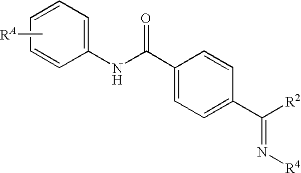

| Comp. | R^{A1} | R² | R⁴ | m.p. (° C.) |
|---|---|---|---|---|
| Ij-1 | 4-tBu | H | OMe | 176-177 |
| Ij-2 | 4-tBu | H | OPh | 146-147 |
| Ij-3 | 4-tBu | H | OtBu | 169-170 |
| Ij-4 | 4-tBu | Me | OMe | 154-155 |

### TEST EXAMPLES

CHO-K1 cells were transfected with human TRPV1 receptors and a cell culture stably expressing the receptor was seeded on a medium of alpha-MEM containing FBS(10%), HEPES (20 mM), geneticin (1 mg/mL) and antibiotic-antimycotic mixed solution(1%) in 96-well plates, and incubated at 37° C. and 5% CO2 for 2-3 days.

The medium was then substituted with Lording Buffer (20 mM HEPES, 115 mM NaCl, 5.4 mM KCl 0.8 mM MgCl₂, 1.8 mM CaCl₂, 13.8 mM D-glucose, 2.5 mM probenecid, pH 7.4) containing Fluo-3 AM (5 μM) and incubated at 37° C. and 5% CO₂ for 1 hour. The test compounds were diluted to different concentrations and added to the plates after the incubated culture was washed with Wash Buffer (20 mM HEPES, 115 mM NaCl, 5.4 mM KCl 0.8 mM MgCl₂, 5.0 mM CaCl₂, 13.8 mM D-glucose, 2.5 mM probenecid, 0.1% BSA, pH 7.4) and placed at room temperature for 15 minutes.

The plate was set up in Functional Drug Screening System(FDSS)-3000(Hamamatsu Photonics K.K.) and amounts of Ca²⁺ influx before and after capsaicin-stimulation were measured through changes in the fluorescence intensity of Fluo-3.

An inhibitory activity was evaluated by measuring fluorescence intensity in the presence of various concentrations of the test compounds and calculating their % inhibition on the basis that the maximum fluorescence intensity is 100% inhibition when stimulated by Wash Buffer, and 0% inhibition when stimulated by capsaicin

Data analysis and calculation of IC₅₀ values were performed with Excel(Microsoft Corporation) and XLfit(idbs). The results were shown below.

| | IC₅₀ (nM) |
|---|---|
| Ia-1 | 176 |
| Ia-2 | 297 |
| Ie-2 | 230 |
| Ib-1 | 210 |
| Ib-2 | 190 |
| Ic-1 | 250 |
| If-1 | 6 |
| If-2 | 160 |
| If-3 | 49 |
| Ii-1 | 39 |
| Ij-4 | 163 |

### Example of Formulation 1

Tablets containing the following ingredients are prepared.

Ingredients:

| | | |
|---|---|---|
| a compound of the formula (I) | 10 | mg |
| lactose | 90 | mg |
| crystalline cellulose | 30 | mg |
| CMC-Na | 15 | mg |
| magnesium stearate | 5 | mg |
| | 150 | mg |

A compound of the formula (I), lactose, crystalline cellulose, CMC-Na(sodium carboxymethylcellulose) are passed through a sieve of 60 mesh and mixed. Magnesium stearate is added to the mixture to obtain a mixed powder for tablets. The mixture is compressed to give a tablet of 150 mg.

### Example of Formulation 2

The ingredients were mixed and heated to give a sterilized injection.

Ingredients:

| | | |
|---|---|---|
| compound of the formula(I) | 3 | mg |
| non-ionic surfactant | 15 | mg |
| purified water for injection | 1 | mg |

### INDUSTRIAL APPLICABILITY

The compound of the present invention has an affinity for TRPV1 receptors and is useful as an analgesic agent.

1. A pharmaceutical composition for modulating TRPV1-receptor function comprising a compound of the formula (I): 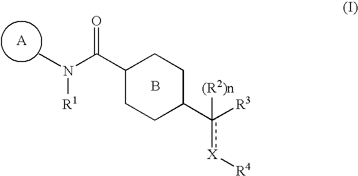
wherein ring A is an optionally substituted monocyclic or bicyclic carbocycle, or an optionally substituted monocyclic or bicyclic heterocycle,
ring B is an optionally substituted benzene ring or an optionally substituted 6-membered heteroaromatic ring containing N atom,
R¹ is hydrogen, optionally substituted lower alkyl or optionally substituted acyl, a dashed line means existence or absence of a bond,
1) when the dashed line means existence, then n is 0, and
1-1) X is ═CR^{x}— or ═N—, and R³ and R⁴ are taken together, with the atoms to which they are bonded, to form an optionally substituted 5-membered non-aromatic heterocycle or optionally substituted 6-membered non-aromatic heterocycle containing at least one heteroatom selected from O and S, and Rx is hydrogen, halogen, lower alkyl, halogeno(lower)alkyl, lower alkoxy, halogeno(lower)alkoxy or acyl, or
1-2) X is ═N—, R³ is lower alkyl, R⁴ is lower alkoxy or aryloxy,
2) when the dashed line means absence of a bond, then n is 1, R² is hydrogen or optionally substituted lower alkyl, X is —O—, —S— or —NR⁵—, and R³ and R⁴ taken together, with the atoms to which they are bound, to form optionally substituted 5-membered non-aromatic heterocycle or optionally substituted 6-membered non-aromatic heterocycle containing at least one heteroatom selected from O and S, R⁵ is hydrogen, lower alkyl, halogeno(lower)alkyl, alkoxy, halogeno(lower)alkoxy or acyl,
a pharmaceutically acceptable salt or a solvate thereof as an active ingredient.

2. The pharmaceutical composition for modulating TRPV1-receptor function as claimed in claim 1 wherein 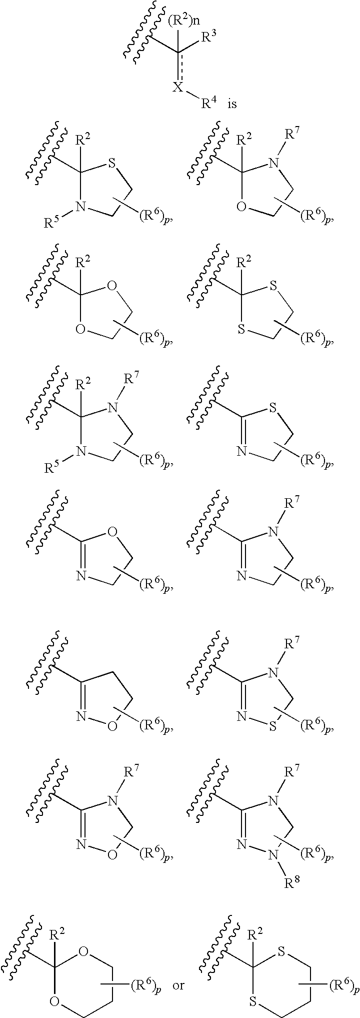
wherein R² is hydrogen or lower alkyl, p is an integer of 0 to 4, R⁶ is hydrogen, halogen, lower alkyl, lower alkoxy, halogeno(lower)alkyl, halogeno(lower)alkoxy or acyl when R⁶ bounds to a constituent carbon atom of the ring,
when p is 2 or more, two of R⁶ taken together may form oxo to attach to a constituent carbon atom or a constituent sulfur atom,
two of R⁶ taken together may form thioxo to attach to a constituent carbon atom,
each of R⁶ may be independently different, and
R⁷ and R⁸ are each independently hydrogen, lower alkyl, lower alkoxy, halogeno(lower)alkyl, halogeno(lower)alkoxy or acyl.

3. The pharmaceutical composition for modulating TRPV1-receptor function as claimed in claim 1 or 2 wherein ring A is optionally substituted benzene, optionally substituted naphthalene, optionally substituted tetrahydronaphthalene, optionally substituted pyridine, optionally substituted pyrimidine, optionally substituted pyridazine, optionally substituted pyrazine, optionally substituted pyrrole, optionally substituted pyrazole, optionally substituted imidazole, optionally substituted triazole, optionally substituted oxazole, optionally substituted isoxazole, optionally substituted thiazole, optionally substituted isothiazole, optionally substituted indole, optionally substituted isoindole, optionally substituted indoline, optionally substituted isoindoline, optionally substituted benzimidazole, optionally substituted benzimidazoline, optionally substituted quinoline, optionally substituted isoquinoline or optionally substituted tetrahydroquinoline.

4. The pharmaceutical composition for modulating TRPV1-receptor function as claimed in claim 1 or 2 wherein ring A is para-substituted benzene.

5. The pharmaceutical composition for modulating TRPV1-receptor function as claimed in claim 1 or 2 wherein R¹ is hydrogen.

6. The pharmaceutical composition for modulating TRPV1-receptor function as claimed in claim 1 or 2 wherein ring B is optionally substituted benzene or optionally substituted pyridine.

7. A compound of the formula (I′): 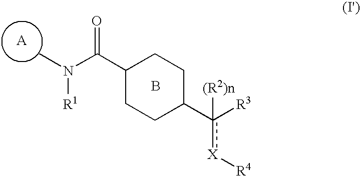
wherein each of symbols is the same as defined in claim 1,
with the proviso that when ring A has substituent(s), then the substituent(s) is(are) at least one group selected from the group of halogen, hydroxy, lower alkyl, halogeno(lower)alkyl, hydroxy(lower)alkyl, lower alkoxy, halogeno(lower)alkoxy, hydroxy(lower)alkoxy, amino, lower alkylamino, acylamino, cycloalkyl, aryl, aryloxy and heterocyclyl,
when the optionally substituted 5-membered non-aromatic heterocycle or optionally substituted 6-membered non-aromatic heterocycle containing at least one heteroatom selected from O and S which are formed by taken together R³ and R⁴, with the atoms to which they are bound, has substituent(s), then the substituent(s) is(are) at least one of group selected from the group of halogen, lower alkyl, halogeno(lower)alkyl, lower alkoxy, halogeno(lower)alkoxy, acyl, oxo and thioxo, and then the non-aromatic heterocycle is not dithiolane,
a pharmaceutically acceptable salt or a solvate thereof.

8. The compound, a pharmaceutically acceptable salt or a solvate thereof as claimed in claim 7 wherein R³ and R⁴ taken together, with the atoms to which they are bound, to form optionally substituted 5-membered non-aromatic heterocycle.

9. A compound of the formula (I″): 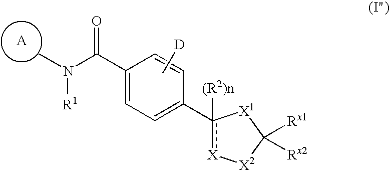
wherein R¹ is hydrogen, optionally substituted lower alkyl or optionally substituted acyl, 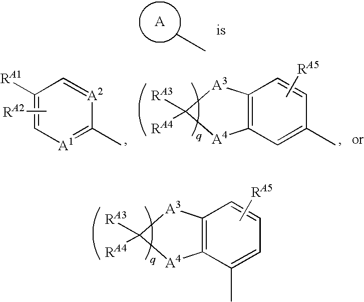
A¹ is N or CR^{A6}, A² is N or CR^{A7},
A³ is NR^{A8} or CR^{A9}R^{A10}, A⁴ is NR^{A11} or CR^{A12}R^{A13},
R^{A1} is lower alkyl, halogeno(lower)alkyl, halogeno(lower)alkoxy or halogen,
R^{A2}, R^{A3}, R^{A4}, R^{A5}, R^{A6}, R^{A7}, R^{A8}, R^{A9}, R^{A10}, CR^{A11}, R^{A12} and R^{A13} are each independently hydrogen, halogen, hydroxy, lower alkyl, halogeno(lower)alkyl, hydroxy(lower)alkyl, lower alkoxy, halogeno(lower)alkoxy, hydroxy(lower)alkoxy, amino, lower alkylamino, acylamino, cycloalkyl, aryl, aryloxy or heterocyclyl,
each of R^{A3} or each of R^{A4} may be independently different,
q is an integer of 1 to 3,
D is hydrogen, halogen, lower alkyl, lower alkoxy or acyl,
dashed line means existence or absence of a bond,
1) when the dashed line means existence of a bond, then n is 0,
X is ═CR^{x}— or ═N—, Rx is hydrogen, halogen, lower alkyl, halogeno(lower)alkyl, lower alkoxy, halogeno(lower)alkyl or acyl;
2) when the dashed line means absence of a bond, then n is 1, R² is hydrogen or optionally substituted lower alkyl, X is —O—, —S— or —NR⁵—, R⁵ is hydrogen, lower alkyl, halogeno(lower)alkyl, lower alkoxy, halogeno(lower)alkoxy or acyl,
X¹ and X² are each independently —O—, —S—, —NR⁵— or —CR^{x3}R^{x4}—,
R^{x1}, R^{x2}, R^{x3} and R^{x4} are each independently hydrogen, halogen, lower alkyl, halogeno(lower)alkyl, lower alkoxy, halogeno(lower)alkoxy or acyl, R^{x1} and R^{x2}, or R^{x3} and R^{x4} taken together may form oxo or thioxo,
each of R⁵, each of R^{x3}, or each of R^{x4} may be independently different,
a pharmaceutically acceptable salt or a solvate thereof.

10. A pharmaceutical composition comprising a compound, a pharmaceutically acceptable salt or a solvate thereof as claimed in any one of claims 7 to 9 as an active ingredient.

11. A pharmaceutical composition for modulating TRPV1-receptor function comprising a compound, a pharmaceutically acceptable salt or a solvate thereof as claimed in any one of claims 7 to 9 as an active ingredient.

12-13. (canceled)

14. A method for modulating TRPV1-receptor function which comprises employing the composition of claim 1 as a TRPV1 receptor-antagonist.

15. A method for modulating TRPV1-receptor function which comprises employing the composition of claim 11 as a TRPV1 receptor-antagonist.

16. A method for modulating TRPV1-receptor function which comprises employing the composition of claim 1 as an analgesic agent.

17. A method for modulating TRPV1-receptor function which comprises employing the composition of claim 11 as an analgesic agent.
